(19) United States
(12) Patent Application Publication
(10) Pub. No.: US 2018/0051316 A1
(43) Pub. Date: Feb. 22, 2018

(54) DETECTION OF NUCLEIC ACID POLYMERASE CONFORMATIONAL CHANGES USING A NANOTUBE

(71) Applicant: THE REGENTS OF THE UNIVERSITY OF CALIFORNIA, Oakland, CA (US)

(72) Inventors: Philip G. Collins, Irvine, CA (US); Gregory A. Weiss, Irvine, CA (US); Yongki Choi, Irvine, CA (US); Tivoli Olsen, Irvine, CA (US)

(21) Appl. No.: 15/537,072

(22) PCT Filed: Dec. 17, 2015
(86) PCT No.: PCT/US15/66321

§ 371 Date: Jun. 16, 2017

Related U.S. Application Data
(60) Provisional application No. 62/093,671, filed on Dec. 18, 2014.

(51) Int. Cl. C12Q 1/48; G01N 27/414

(52) U.S. Cl. CPC C12Q 1/485; G01N 2333/9126; C12Y 207/07007; G01N 27/4146

(57) ABSTRACT

The invention provides methods and compositions for detecting a change in a nucleic acid polymerase conformation involving contacting a nucleic acid polymerase non-covalently attached to a single walled carbon nanotube (SWNT) with a first nucleotide or first nucleotide analog and a template and detecting the conformationally changed nucleic acid polymerase by measuring a first electrical conductance change in the SWNT between the nucleic acid polymerase and the conformationally changed nucleic acid polymerase. The method is useful for sequencing of polynucleotides.

### CROSS-REFERENCES TO RELATED APPLICATIONS

This application claims the benefit of U.S. Provisional Application No. 62/093,671, filed Dec. 18, 2014, the content of which is incorporated hereby by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under Grant No. 1 RO1 CA133592-01, awarded by the National Institutes of Health, and Grant No. ECCS-1231910 awarded by the National Science Foundation. The Government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48538-526001WO_ST25.TXT, created Dec. 13, 2015, 2,828 bytes, machine format IBM-PC, MS-Windows operating system, is hereby incorporated herein by reference in its entirety and for all purposes.

### BACKGROUND

Within the industry of DNA sequencing, the use of synthetic (non-natural) molecules is a primary strategy for differentiating between the four nucleotide bases (A, C, T, and G) that make up DNA. This strategy was applied successfully to the venerable method of Sanger sequencing, which was used for the original human genome effort.

Technologies exist for sequencing DNA, but there is commercial demand for new techniques that can increase speed, decrease error-rates, and reduce complexity, costs, and reagent requirements. There is significant interest in technologies that can sequence DNA using electronic circuits, since solid state electronics can offer many benefits in speed, cost, and complexity.

In recent years, electronic architectures have generated that operate by passing DNA through a nanopore and monitoring the ionic current through the same pore or by passing DNA through a nanopore but transducing the transit using an adjacent electrical tunnel junction. Both platforms rely on DNA passage through nanopores, so they share characteristic difficulties of working with nanopores such as instability, fragility, and precise fluid handling requirements. Furthermore, the passage of DNA through nanopores has limited signal-to-noise so that, in practice, any sequencing information must be independently confirmed using fluorescence methods. Additionally, a high error-rate and “slip” through the nanopore limits applications, such as for sequencing highly repetitive sequences of short tandem repeats, required for human identification applications.

A biosensor is an analytical device that incorporates a biological recognition element in direct spatial contact with a transduction element. That integration ensures the rapid and convenient conversion of biological events to detectable signals. Among diverse electrical biosensing architectures, devices based on field-effect transistors (FETs) have attracted great attention because they are a type of biosensor that can directly translate interactions between target molecules (e.g., biological molecules) and the transistor surface into readable electrical signals. In a standard field effect transistor, current flows along a conducting path (the channel) that is connected to two electrodes, (the source and the drain). The channel conductance between the source and the drain is switched on and off by a third (gate) electrode that is capacitively coupled through a thin dielectric layer. Field-effect transistors detect target chemicals and measure chemical concentrations for a wide range of commercial applications including, for example, industrial process control, leak detection, effluent monitoring, and medical diagnostics.

For example, disclosed in U.S. patent application Ser. No. 13/626,760 is an electronic device that is sensitive enough to detect at the single molecule level. Aspects of the invention are accomplished using an electrically-conducting channel that has a single sensitizing molecule attached thereto. Accordingly, devices disclosed therein monitor the dynamics of a single molecule reaction, and can be used in important single molecule biochemical assays, such as detectors in a single molecule sequencing reaction.

Thus, there is a need in the art for next generation DNA sequencing techniques that are more efficient and more informative than existing techniques. Provided here are solutions to these and other problems in the art.

### BRIEF SUMMARY

Provided herein, inter alia, are circuits with a mixture of natural and unnatural nucleotide bases to determine the genetic sequence of a DNA sample. Described are specific techniques and reduction to practice of using the circuit to determine the genetic code of a strand of DNA.

The circuit enables sequencing of DNA and, by extension, sequencing of RNA and carbohydrates. The invention offers a method of low cost, high speed, high fidelity DNA sequencing that could successfully compete with more traditional sequencing methods.

The methods and compositions provided herein may follow the activity of single-molecules during enzymatic processing. Synthetic substrates, nucleotides, and fluorophores may be used to generate unique and distinguishable signals from a strand of DNA

### BRIEF DESCRIPTION OF THE DRAWINGS

FIGS. 1A-1C. Electrical monitoring of KF activity with chemically modified dNTPs. FIG. 1A: A single KF nanocircuit and the chemically modified dNTPs tested for their incorporation by KF. (a) A schematic diagram of a single-walled carbon nanotube field effect transistor (SWCNT-FET) non-covalently bioconjugated to a single molecule of DNA polymerase I (KF) through a single cysteine introduced in the “fingers” subdomain. A pyrene-maleimide linker (yellow) adhered to the SWCNT-FET through 7E-7E stacking and covalently attached to the single cysteine to immobilize the KF. The SWCNT-FET was grown on SiO₂, connected to source and drain metal electrodes, and passivated with a polymer (PMMA, red).

FIG. 1B: Atomic force microscopy shows the 1-2 nm diameter of the SWNT FET with a single KF attachment (7 nm, arrow). FIG. 1C: Chemical structures of analog dNTPs disclosed herein. Chemical modifications from the native dNTPs are highlighted.

FIG. 2A-2F. Changes in the current during native and analog dNTP incorporation. FIG. 2A: In this current measurement in the presence of poly(dC)₄₂ template and its complementary native dGTP, ΔI(t) excursions occur during each base incorporation. High and low current states correspond to the enzyme's open and closed conformations, respectively. FIGS. 2B-2F: Time magnification of the data corresponding to FIG. 2A (time window 1.5 to 2.5 s) illustrates the decrease in switching events corresponding to base incorporation of dGTP (FIG. 2B), α-thio-dGTP (FIG. 2C), 6-chloro-2APTP (FIG. 2D), and 2-thio-dCTP (FIGS. 2E-2F). To the right of each of FIGS. 2B-2F, the magnified view depicts a single ΔI(t) excursion for each indicated base highlighting the single base resolution, with bar indicating 1 ms time interval.

FIGS. 3A-3B. Direct comparison of the probability distributions of open, τₒₚₑₙ, and closed, τclosed, states durations during incorporation of the indicated dNTPs from >50 s data sets. Y-axes plotted as log probability %. For both τ_{closed} (FIG. 3A) and τₒₚₑₙ (FIG. 3B), the homopolymeric poly(dC)42 provided the template. In FIGS. 3A-3B, single-exponential fits for each nucleotide are shown as solid lines.

FIGS. 4A-4B. Electronic signal generated in the processing of poly(dA)₄₂. FIG. 4A: When KF processes poly(dA)₄₂ in the presence of the natural nucleotide deoxythymidine triphosphate (dTTP), each base pair incorporation produces a negative current spike ΔI<0. FIG. 4B: When dTTP is replaced by the unnatural nucleotide 2-thio-2′-deoxythimidine-5′-triphosphate (2-thio-dTTP), base incorporations produce positive current spikes ΔI>0.

FIGS. 5A-5C. Electronic signals generated in the processing of heterogeneous substrates. FIG. 5A: When KF processes heterogeneous substrates in the presence of all four natural nucleotides (dNTP), each base pair incorporation produces a negative current spike ΔI<O. Individual spikes can be enumerated as shown, but in general they do not differentiate one type of base from another. FIG. 5B: FIG. 5B demonstrates simulation of the same data set with dTTP replaced by 2-thio-dTTP. With the thiolated deoxythymidine, positive spikes now indicate (#2, 6, 7) the locations where T nucleotides were incorporated. FIG. 5C demonstrate that when KF processes heterogeneous substrates in the presence of natural nucleotides (dNTP) mixed with certain analogs, the resulting pattern contains positive and negative current spikes that can be used to identify a chosen base. This example shows data acquired using three native nucleotides (dATP, dTTP, dCTP) mixed with 6-Cl-2APTP as an analog for G incorporations. This information is used in a method for nanotube sequencing of an oligonucleotide.

FIG. 6. Figure depicts representative 15% SDS-PAGE gel of KF after over-expression and purification. KF was purified to >95% homogeneity and migrated at its expected mass of about 68 kDa.

FIG. 7. Figure depicts fluorescence-based activity assay depicting KF(L790C) (black circles) and wild-type KF (gray circles) activity under steady-state conditions. The primer extension reaction occurs in the presence of dATP, dTTP, dCTP, and dGTP. The raw data was subtracted from background, which measured activity in the absence of dNTPs.

FIGS. 8A-8B. Figures depict ensemble assay showing incorporation of dNTP analogs with templates described herein. Polymerization products with dNTP analogs and the A/T incorporation template (FIG. 8A) or the G/C incorporation template (FIG. 8B) were electrophoresed on a 5% high-resolution agarose gel. Negative control reactions with only 3 dNTPs, omitting dTTP (1), dATP (2), dCTP (8), and dGTP (9), contained no dsDNA. Positive control reactions with all four dNTPs showed conversion to dsDNA with both the A/T incorporation template (3) and the G/C incorporation template (10). Reactions with dNTP analogs (4-7 and 11-14) omitted their native dNTP counterpart and contained the remaining 3 native dNTPs. Opposite the A/T incorporation template, α-thio-dTTP (4) and 2-thio-dTTP (5) incorporated opposite the template base A, and α-thio-dATP (6) and 6-Cl-2APTP (7) incorporated opposite the template base T. Opposite the G/C incorporation template, α-thio-dCTP (11) and 2-thio-dCTP (12) incorporated opposite the template base G, and α-thio-dGTP (13) and 6-Cl-2APTP (14) incorporated opposite the template base C. After visualization, the image colors were inverted, then changed to black and white.

### DETAILED DESCRIPTION OF THE INVENTION

Provided herein, inter alia, is a method of detecting a change in a nucleic acid polymerase confirmation; a method of sequencing a nucleic acid polymerase in which the conformation change of a nucleic acid polymerase is detected. In embodiments, the methods include detecting conformational change of a nucleic acid polymerase with nucleic acid analogs.

### Definitions

The following definitions are included for the purpose of understanding the present subject matter and for constructing the appended patent claims. Abbreviations used herein have their conventional meaning within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The term “hybridization” and the like refer, in the usual and customary sense, to formation of double stranded (i.e., duplex) nucleic acid, including e.g., DNA/DNA hybrid, DNA/RNA hybrid, and RNA/RNA hybrid. It is understood that formation of duplex nucleic acid can be through Watson-Crick base-pairing. The phrase “selectively (or specifically) hybridizes to” refers to the binding, duplexing, or hybridizing of a nucleic acid to a particular nucleotide sequence with a higher affinity, e.g., under more stringent conditions, than to other nucleotide sequences (e.g., total cellular or library DNA or RNA).

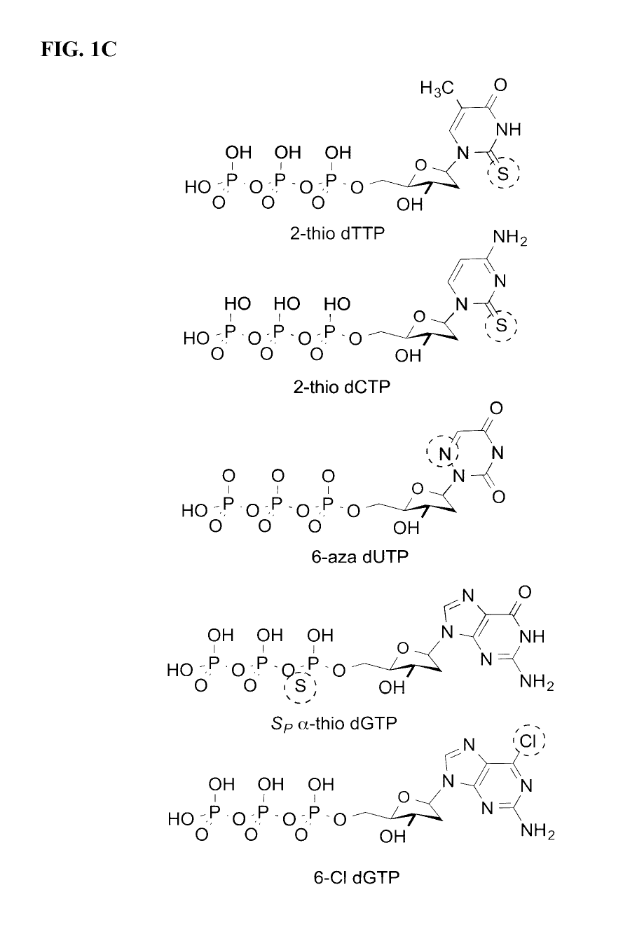

As used herein, the conformation change of a nucleic acid polymerase is detected using a single walled carbon nanotube field-effect transistor (SWCNT-FET). For example, a Klenow Fragment (KF) nanocircuit includes a SWCNT-FET noncovalently bioconjugated to a single molecule of DNA polymerase I (KF) through a single cysteine introduced in the “fingers” subdomain. The conformation change is measured by ΔI(t) signals produced by the KF nanocircuit. The device produces uninterrupted sequences of negative ΔI(t) excursions, each indicate the formation of one basepair, and with an inverted amplitude reflects a different KF conformation (FIG. 2C, FIG. 2F).

As used herein, in embodiments, the first nucleotide or a first nucleotide analog may be the same as a second nucleotide or a second nucleotide analog, respectively. In embodiments, the first nucleotide or a first nucleotide analog may be different from a second nucleotide or a second nucleotide analog, respectively.

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent hybridization conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

In embodiments, the single molecule sensing device 10 may take the form of a transistor, namely, a field effect transistor (FET) with the attached biomolecules serving as a “gate” to an electrical circuit. In this embodiment, a single sensitizing molecule services a single molecule gate for the device. The transistor embodiment may include a two or three terminal transistor. The conduction channel may also be formed from metals, metal oxides, semiconductors, or nanometer-scale conductors such as nanowires, graphene, or single-walled carbon nanotubes (SWNTs). In one embodiment, the conduction channel is a single SWNT.

### Methods

Provided here is a method of detecting a change in a nucleic acid polymerase conformation. The method includes contacting a nucleic acid polymerase non-covalently attached to a single walled carbon nanotube (SWNT) with a nucleotide or nucleotide analog (e.g. a first nucleotide or nucleotide analog) and a template nucleic acid sequence (e.g. sense strand oligonucleotide or polynucleotide) thereby forming a conformationally changed nucleic acid polymerase bound to the nucleotide or nucleotide analog and the template nucleic acid sequence. The conformationally changed nucleic acid polymerase is detected by measuring a change in the electrical conductance of the SWNT between the nucleic acid polymerase and the conformationally changed nucleic acid polymerase. The term “contacting” and the like refer, in the usual and customary sense, to the bringing of two or more species into sufficiently close contact that an interaction can occur between the species, e.g., binding, chemical reaction, or the like. The term “electrical conductance change” and the like refer, in the usual and customary sense, to a change in electric conductance that can be measured by methods known in the art and disclosed herein. The term “conformationally changed nucleic acid polymerase” and the like refer, in the usual and customary sense, to a change in the secondary, tertiary and/or quaternary structure or a nucleic acid, as known in the art.

As disclosed herein, the change in conductance may be the result of a change in the position of a sensitizing molecule (e.g. an amino acid) that forms part of the nucleic acid polymerase relative to the nucleic acid polymerase and the conformationally changed nucleic acid polymerase. The current fluctuations can consist of simple increases and decreases in a square-edged pattern. Alternately, the fluctuations can comprise any wavelet including shapes that are triangular, sinusoidal, or having any number of Fourier components. The amplitudes, durations, and shapes of these wavelets all encode the activity of the target-specific component and therefore can be analyzed using a computer to uncover the kinetics of the binding and other mechanical and electronic degrees of freedom. Statistical analysis of these parameters provides insight into the kinetic variability, transitions, and intermediate chemical states of the target binding and unbinding processes. The degrees of freedom in the current signal distinguish among multiple similar target molecules that all bind to the same site, for example between a target molecule and an inhibitor molecule of the binding site. These degrees of freedom can also distinguish weak interactions such as molecule recognition that occur before true binding.

The method of detecting a change in a nucleic acid polymerase conformation may be used as part of a method of sequencing a nucleic acid (e.g. DNA or RNA). Thus, in some embodiments, the method further comprises, after detecting the conformationally changed nucleic acid polymerase bound to the first nucleotide or nucleotide analog, detecting a second change in conformation of said nucleic acid polymerase by allowing said conformationally changed nucleic acid polymerase to release the first nucleotide or nucleotide analog thereby reforming the nucleic acid polymerase. The method then includes contacting the nucleic acid polymerase non-covalently attached to a single walled carbon nanotube (SWNT) with a second nucleotide or nucleotide analog thereby forming a conformationally changed nucleic acid polymerase bound to the second nucleotide or nucleotide analog. The conformationally changed nucleic acid polymerase bound to the second nucleotide or nucleotide analog is detected by measuring a change in the electrical conductance of the SWNT between the nucleic acid polymerase and the conformationally changed nucleic acid polymerase.

In embodiments, the first and/or second nucleotide or nucleotide analog produce unique conductance signal that is detected. The unique conductance signal is used to identify said first and/or second nucleotide or nucleotide analog thereby identifying the sequence of the template nucleic acid. The terms “conductance signal,” “first conductance signal,” “unique conductance signal” and the like refer, in the usual and customary sense, to the conductance of a species as measured by methods known in the art including methods disclosed herein.

Useful in the methods provided herein are carbon nanotube circuits that can operate faster, at low cost, and at a potentially much lower error-rate than more traditional sequencing technologies. The compositions and methods provided herein offer significant improvement over both of the nanopore-based electronic architectures. First, the carbon nanotube circuit generates an electronic signal with excellent noise characteristics that does not need independent confirmation. Second, the nanotube circuit tolerates a wide range of environments and rough handling, such that specifications on fluid handling and overall system complexity can be significantly relaxed compared to nanopore architectures. Third, the nanotube circuit is conceptually straightforward and easily adapted to operate in a variety of different modes. Fourth, the approach may employ a high fidelity enzyme to provide base pair discrimination; estimated error rates could be as low as the theoretical maximum for the enzyme of 18×10⁻⁶. Such low error-rates would represent an approximately 10,000-fold improvement over currently available, commercial instruments. Thus, provided herein are methods and composition that significantly reduce cost, complexity, error-rates, and the added burden of extensive re-sequencing. A general description of the nanotube circuits are provided in Appendix A and in U.S. patent application Ser. No. 13/626,760.

The invention generally provides an electronic device that is sensitive enough to detect at the single molecule level. Aspects of the invention are accomplished using an electrically-conducting channel that has a single sensitizing molecule attached thereto. Accordingly, devices of the invention monitor the dynamics of a single molecule reaction, and can be used in important single molecule biochemical assays, such as detectors in a single molecule sequencing reaction.

Any type of conduction channel that is generally found in field effect transistors can be used with the invention. Exemplary conduction channels are formed from metals, metal oxides, semiconductors, or nanometer-scale conductors such as nanowires, graphene, or single-walled carbon nanotubes (SWNTs). In embodiments, the conduction channel is a single SWNT.

As a class of materials, SWNTs are semiconductors with electronic bandgaps that can vary from 1 electron volt to effectively zero. This variation leads to the classification of carbon SWNTs as metallic or semi-metallic, and others as semiconducting. With the aid of connecting electrodes, electrostatic gates, and other control circuitry, semiconducting SWNTs can be configured as sensor FETs, as RF amplifiers, or as low-temperature single electron transistors. The device and method does not preclude such additions, because in embodiments the device is composed of only a two-terminal, SWNT conductor. SWNTs are conduction channels in which single molecule sensing devices can be fabricated from SWNT wires of any type, with or without gate electrodes, and on glass, plastic, or silicon substrates. The single molecule sensing device described here can be one component within a FET or any number of more complex electronic or opto-electronic devices and circuitry.

One aspect of the disclosure is the reliable achievement of only one active sensitizing molecule in each device. In general, sensitizing molecules will coat a SWNT with a mean spacing that is determined by the concentration and incubation period used in preparation. Once that mean spacing has been empirically determined for a particular set of conditions, the SWNT conductor can be defined by lithography to have an equal length. In practice, this length is typically 1 to 100 nm when sensitizing molecules directly attach to the SWNT conductor, a range that is a difficult to control using optical lithography.

In embodiments, linker molecules serve as an attachment intermediary that improves the control over the mean separation of sensitizing molecules. Any method known in the art may be used to attach the single sensitizing molecule to the conductor. In embodiments, a linker molecule is used to attach the single sensitizing molecule. In embodiments, the linker molecule includes at least a first and a second functional group. Generally, the first functional group interacts with the conduction channel (e.g., the single-walled carbon nanotube) and the second functional group interacts with the sensitizing molecule. Exemplary first functional groups include a pyrene, a benzene, a cyclohexane, and 2,3-dichloro-5,6-dicyano-1,4-benzoquinone. An exemplary second functional group is maleimide. In certain embodiments in which the conduction channel is a SWNT, the linker molecule interacts with a sidewall of the SWNT through pi-pi stacking.

Using linkers, the length between sensitizing molecules can be dramatically increased up to 1 micrometer or more. With sensitizing molecules spaced 1 micrometer apart, it becomes possible to use standard lithographic masking techniques to define wafers full of conductors, each approximately 1 micrometer in length. Alternately, given a desired device pitch as set by the mask design, the concentration of sensitizing molecules and duration of incubation can be varied to achieve the same result of one molecule per device. The single molecule sensing devices can be produced in at least 8 out of 10 fabrication attempts, all without disrupting the sp2 character of a SWNT conductor.

Any sensitizing molecules known in the art can be used with devices of the invention, and the sensitizing molecule chosen will depend on the molecule to be detected or the reaction to be monitored. Exemplary sensitizing molecules include an enzyme, a protein, a nucleic acid, a ribozyme, an aptamer, and a polysaccharide. In certain embodiments, the enzyme is a lysozyme, a protein kinase A, or a DNA Polymerase I.

In other aspects, more than one sensitizing molecule may be necessary in each device to achieve single molecule dynamic sensing. For example, at a desired operating temperature or pH, a particular type of sensitizing molecule might only have a 25% probability of being chemically active. Under these conditions, it is appropriate to attach additional sensitizing molecules (e.g., four) to each conductor in order to produce a device in which one is likely to be active. This higher density of attachments is readily achieved using the scheme described above, either by increasing the length of the devices to an appropriate multiple of the mean separation distance between molecules, or else by decreasing the same separation by modifying the attachment conditions.

In embodiments, the single molecule sensing device includes multiple conductors in parallel (e.g., SWNT conductors). A single active sensitizing molecule is attached to one of the conductors, and it contributes a dynamic electronic signal that is separable from the parallel but static conductance of the unmodified conductors. This embodiment provides additional flexibility in the design of the conductor synthesis or placement, and in the successful fabrication of single molecule sensing devices using sensitizing molecules that have very low attachment probabilities.

In embodiments, multiple single molecule sensing devices are fabricated in parallel using the same type of sensitizing molecule, with one sensitizing molecule attached per device. In another embodiment, multiple conductors are prepared and then exposed to different sensitizing molecules, in order to achieve multiple single molecule sensing devices that are sensitized towards differing targets. In another embodiment, the single molecule sensing device responds to multiple targets through a sensitizing molecule with a range of specificities.

In embodiments, a single molecule sensing device includes a first electrode, and a second electrode. A single-walled carbon nanotube is connected, respectively, to the first electrode and the second electrode. The device includes at least one linker molecule having first and second functional groups, the at least one linker molecule having the first functional group non-covalently functionalized with a sidewall of the single-walled carbon nanotube. A single sensitizing molecule having at least one functional group, said at least one functional group of the single sensitizing molecule being functionalized with the second functional group of the at least one linker molecule.

In embodiments, a method for making a single molecule sensing device includes forming at least one single-walled carbon nanotube on a substrate that is connected to a first electrode and a second electrode; non-covalently functionalizing the single-walled carbon nanotube sidewall of the device with at least one functional group of at least one linker molecule containing a plurality of functional groups; and functionalizing at least one of the functional groups of the at least one linker molecule with one or more functional groups of a single sensitizing molecule.

In embodiments, a method of using a single molecule sensing device having a single-walled carbon nanotube (SWNT) is disclosed. The SWNT is disposed on a substrate and connected to a first electrode and a second electrode, the sensing device having a single sensitizing molecule secured to the SWNT using a linker molecule non-covalently functionalized with the SWNT. Voltage is applied across the SWNT. The sensitizing molecule is exposed to a chemical environment. Fluctuations in the current flowing through the SWNT are monitored.

In embodiments, methods for sequencing a nucleic acid using a single molecule sensing device is disclosed. The sensing device includes a conductive channel. The conductive channel may include a single-walled carbon nanotube (SWNT) on a substrate connected to a first electrode and a second electrode. The sensing device has a single sensitizing enzyme secured to the channel using a linker molecule non-covalently functionalized with the channel (e.g., SWNT). The method includes exposing the device to at least one type of nucleotide; applying a voltage potential across the channel; monitoring fluctuations in the current flowing through the SWNT; and identifying the nucleotides incorporated into a nucleic acid template by the enzyme based at least in part on the monitored fluctuations in current. The enzyme may be a polymerase or a reverse transcriptase. The nucleotide may be a nucleotide analog. In certain embodiments, the device is exposed to more than one type of nucleotide at a single time.

The sensing device may also be used to determine processing kinetics of a protein or enzyme. Still another application of the sensing device is to determine the effects of a genetic mutation. Devices using sensitizing molecules or targets with genetic mutations can be compared to the performance obtained from similar devices with sensitizing molecules or targets that do not have the mutation. In still another application, the sensing devices can be used to measure the effects of drugs or other small molecules on a protein, either to make it active or inactive.

Method of fabricating devices of the invention may involve a biochemical conjugation protocol followed by controlled rinsing. Such a process results in devices of the invention having one sensitizing molecule and no nonspecific binding of interfering molecules. In certain embodiments, the sensitizing molecule is directly attached to the conductor through a non-covalent interaction. In other embodiments the sensitizing molecule is attached to an intermediate linker molecule having at least two functional groups, one designed for the non-covalent attachment and the other for versatile bio-conjugation to a sensitizing molecule. One scheme of using an intermediate linker provides a chemically versatile platform for building devices of the invention from a wide class of sensitizing molecules.

In embodiments, a method for making a single molecule sensing device includes forming at least one single-walled carbon nanotube on a substrate that is connected to a first electrode and a second electrode, non-covalently functionalizing the single-walled carbon nanotube sidewall of the device with at least one functional group of at least one linker molecule containing a plurality of functional groups; and functionalizing at least one of the functional groups of the at least one linker molecule with one or more functional groups of a single sensitizing molecule.

In embodiments, the single molecule sensing device may take the form of a transistor, namely, a field effect transistor (FET) with the attached biomolecules serving as a “gate” to an electrical circuit. In this embodiment, a single sensitizing molecule services a single molecule gate for the device. The transistor embodiment may include a two or three terminal transistor. The conduction channel may also be formed from metals, metal oxides, semiconductors, or nanometer-scale conductors such as nanowires, graphene, or single-walled carbon nanotubes (SWNTs). In one embodiment, the conduction channel is a single SWNT.

Generally, the length of the SWNT may vary from about 0.1 to about 10 micrometers. The particular length of the SWNT is chosen such that statistically, a majority of the devices 10 that are manufactured have only a single sensitizing molecule associated with the SWNT. Even more preferably, the length of the SWNT that is exposed to the external chemical environment is chosen such that more than 75% of the devices that are manufactured include only a single sensitizing molecule associated with the SWNT. In some instances, this distance is the distance between the first electrode and the second electrode.

The first electrode and the second electrode may be optionally covered with a cover. The cover may include a window, recess, slot, or other open segment that provides access from the external environment to the SWNT. In this regard, the SWNT can be exposed to a chemical environment. For example, an exposed window can be defined in the cover during the manufacturing process. The protective covering ensures that the majority of the surface including the first and second electrodes is protected from the environment. Moreover, in a preferred embodiment, the length of the window is tailored to achieve the correct device length. The length of the window can be varied to achieve the desired active region on the SWNT. For example, the first and second electrodes may be connected to the SWNT and separated by a distance of 2 μm. The window, however, can be made smaller than the inter-electrode distance. The exposed window within the protective covering exposes the SWNT and the attached sensitizing molecule to the chemical environment. The protective cover can be any electrically-insulating film composed of one or more layers. The film materials include polymers, aluminum oxide, halfnium oxide, silicon dioxide, or silicon nitride. The window is defined within the protective covering using lithographic techniques. Lithographic techniques are well known in the art and comprise using any acceptable combination of optical exposure, electron beam methods, and positive or negative resists.

In embodiments, the device fabrication includes coating devices in a protective covering of positive electron beam resist such as polymethyl methacrylate (PMMA); writing lithographic patterns with an electron beam; and then developing the written areas to expose an active SWNT channel 0.5 to 1.0 μm in length. In another embodiment, device fabrication comprises coating devices in a protective covering of aluminum oxide; coating devices further in a film of optical photoresist; exposing the desired windows to light; developing the written areas to expose narrow windows of the aluminum oxide; etching the aluminum oxide to further expose the underlying SWNT channels 0.5 to 1.0 μm in length. Combinations of two or more layers of materials in the protective coating provide coatings having different chemical properties.

The device is coupled to electronic circuitry. The electronic circuitry is used to both apply a voltage bias (e.g., 50-100 mV) between the first electrode and the second electrode and is also configured to measure the current flow across the SWNT as a function of time. Electronic circuitry may be coupled to a computer 24 having one or more processors therein that is used to control the application of voltage and current through the device as well as acquire, store, and analyze data generated by the device. During operation of the device, a voltage (e.g., constant DC voltage or combination of AC and DC voltages) is applied between the first electrode and the second electrode. The current then passing through the SWNT is measured using electronic circuitry, which may include a current meter with one or more amplifiers.

The first electrode, second electrode, and the SWNT may be disposed atop a substrate. The substrate may include any number of substrate materials such as glass, plastic, or silicon. One alternative embodiment of the invention involves fabrication of the device on an optically transparent substrate such as glass or quartz. Unlike sensor FETs and much of the prior art related to sensing, the device does not require a gate electrode or a conductive supporting substrate. Consequently, the device can be fabricated on a wide range of surfaces including transparent ones. Quartz is preferred for the CVD fabrication process described above because it is compatible with high temperatures. Glass wafers can also be used if the SWNTs are synthesized and deposited onto the substrate by other means, such as spin coating from solution, or if the devices are fabricated on wafers and then transferred to the glass for support. In any case, the use of quartz, glass, sapphire, or other transparent substrate enables optical monitoring of the device. Monitoring the fluorescence signal from tethered molecules is well known in the art, and it is best accomplished through a transparent substrate. A device 10 formed on a quartz substrate allows independent monitoring of molecule dynamics using the electrical techniques described herein and by optical techniques including single molecule fluorescence and smFRET.

In embodiments, electrical and optical signals from the same single molecule is acquired, either at different times or simultaneously. A single molecule sensing device located on a transparent substrate (e.g., quartz) provides a unique opportunity not found in the prior art to complement smFRET with an independent single molecule technique. In this embodiment, the SWNT is illuminated through the transparent substrate using an illumination source. Fluorescent light that is emitted can be collected using an objective lens that uses oil or water to contact the transparent substrate. Fluorescent light can be directed to a photon counter using, for example, a beam splitter.

Such dual-mode monitoring can calibrate the measurements made by one approach, such as the electronic monitoring with turnover measurements of fluorescence made at the ensemble level. Simultaneous interrogation of one molecule by two independent means provides the opportunity to study two different portions of the same molecule, for example to compare a portion that moves, a portion that accepts the transfer of charge, a portion that contains a catalytic site, or a portion that absorbs or emits photons. Synchronous monitoring of two such portions can determine the relative timing and causality of two events, such as the movement of the active site correlated with the conformational changes of a regulatory site. Furthermore, the transparent substrate allows light-induced activation of a catalytic site functionality or a light driven charge-transfer for examination of the resultant conformational change. The SWNT may, in one embodiment, be integrated within a flow cell or the like such that a fluid can flow over the SWNT for measurements. Alternatively, fluids may be selectively deposited on top of the device.

The device may include one linker molecule containing one or more functional groups non-covalently attached to the external sidewall of a SWNT. Functional groups may include pyrene, benzene, cyclohexane, and 2,3-dichloro-5,6-dicyano-1,4-benzoquinone. Functional groups which non-covalently attach to the external sidewall of a SWNT are well known in the art and the specific design for this functional group can comprise any design suitable for use in the present invention. Furthermore, the linker molecule(s) contains one or more functional groups functionalized with another functional group which is or has been attached to the sensitizing molecule in such a way as to maintain some or all of the functionality of the sensitizing molecule. Pairs of functional groups may include an azide and an alkyne, a NHS ester and an amine, a thiol and an alkyne, and a thiol and a maleimide. Functional groups which functionalize with other functional groups are well known in the art and the specific design for this functional group can comprise any design suitable for use in the present invention.

The device may include a single sensitizing molecule containing one or more functional groups functionalized with one or more functional group of one of the linker molecule in such a way as to retain the functionality of the sensitizing molecule. Sensitizing molecules of the present invention include any molecule. Preferable sensitizing molecules include molecules that are chemically specific in their interactions with other molecules. More preferably, sensitizing molecules may include polymers, proteins, DNA, RNA, ribozyme and/or aptamer, polysaccharide, or other biomolecule. Sensitizing molecules 30 are well known in the art and can comprise any sensitizing molecule suitable for use in the present invention.

In embodiments, the linker molecule may include a first functional group that adheres non-covalently to the wall of the SWNT and a second functional group that is designed to attach to the sensitizing molecule. The use of the linker molecule avoids the difficulty of designing an effective, direct attachment between the sensitizing molecule and the SWNT. In this embodiment, the linker molecule and the sensitizing molecule are effectively a single entity. In practice, achieving and controlling the desired surface density often requires that the linker molecule(s) and sensitizing molecule be prepared as two separate solutions, with the final linkage between them performed in place on the SWNT. The sensitizing molecule may include a first functional group and a second functional group which may include a target-selective functional group. The first functional group of the sensitizing molecule binds to the second functional group of the linker molecule. The binding can be any chemical interaction known in the art, for example, covalent or non-covalent binding. In embodiments, the binding is through a covalent bond. The second functional group is designed to bind to a target molecule or multiple target molecules by any binding interaction. The sensitizing molecule also includes a conductivity-modulating component that is ideally located near the site of the SWNT attachment. The conductivity-modulating component need not be in close proximity to the second functional group but the two should communicate through mechanical, allosteric or electronic means, so that interactions of the sensitizing molecule with the chemical target induce dynamic changes in the conductivity-modulating component of the same sensitizing molecule to affect electronic changes in the SWNT.

In embodiments, a pyrene functional group non-covalently may attach to a SWNT surface through pi-pi stacking. A single sensitizing molecule may be associated with the SWNT. Typical electrical characteristics of a completed device may be measured with aqueous electrolyte in direct contact with the sidewall of a semiconducting SWNT.

In embodiments, all three components are combined in a single sensitizing molecule. For example, one amino acid of a protein might be an effective site for binding to a SWNT, another amino acid might have a net surface charge that can modulate the SWNT conductivity, and a third amino acid might serve as a recognition or binding site for the protein's binding partner, the target molecule to be detected. Alternately, a covalent or non-covalent complex can be designed and synthesized to bring all three components together as a single sensitizing agent.

In embodiments, the different functional components of the sensitizing molecule are split among two or more molecules, all of which are covalently or non-covalently assembled on the SWNT conductor. In this alternative embodiment, the conductivity-modulation component can be a molecule that attaches to one functional group of a linker molecule, and the target-selective chemical component can be a second molecule that attaches to a different functional group of the same linker. Alternately, the target-selective chemical component can have a functional group that binds directly to the molecule that contains the conductivity-modulating component. This binding can be through a covalent bond or through non-covalent recognition or docking common to many biomolecules. In every case, some form of mechanical, steric or electrical communication will be achieved between the components, so that the dynamics of the target-specific chemical component result in changes to the conductivity-modulating component of the whole sensitizing complex.

An embodiments, the single molecule sensing device may include a conductor having one or more SWNTs; one or more linker molecules containing two or more functional groups, of which one or more is non-covalently bound to the surface of a SWNT; and a single sensitizing molecule which contains at least one functional group which is functionalized to at least one functional group of a linker molecule.

In embodiments, single molecule sensing device includes a linker molecule containing a carboxylate group and the sensitizing molecule containing an amine. The carboxylate functional group of the linker molecule can be activated as a reactive ester and amidated using techniques that are well known in the art. The reactive ester can then be covalently coupled to an amine group of the sensitizing molecule to form a stable amide bond in a way which is well known in the art.

In embodiments, a single molecule sensing device include a linker molecule which is a pyrene maleimide and the sensitizing molecule containing a reactive thiol group. The maleimide functional group of the linker molecule may be covalently coupled with the thiol group of the sensitizing molecule to form a stable thioester bond in a way which is well known in the art.

In embodiments, a non-covalent single molecule sensing device includes a linker molecule, which is pyrene maleimide and the sensitizing molecule is a protein. Further embodiments include those in which the protein is an enzyme. In embodiments, the enzyme is DNA polymerase or a Reverse Transcriptase. Similar yields of single molecule sensing devices utilizing each of these enzymes have been achieved by tailoring the solution pH, soak duration, and rinse conditions used during attachment of the enzyme.

In embodiments, the sensitizing molecule is a nucleic acid (e.g., DNA, RNA), ribozyme, aptamer, polysaccharide, or other biomolecule. Any sensitizing molecule which undergoes an alteration in conformational dynamics upon binding of or acting upon a substrate or ligand is suitable for use in the present invention. In embodiments the linker molecule comprises a linker molecule containing at least one functional group which is known in the art to non-covalently functionalize to the surface of a SWNT and at least one functional group being a functional group which is known in the art to form bonds with another functional group.

An embodiment is the use of a DNA or RNA polymerase or a Reverse Transcriptase as the single sensitizing molecule non-covalently attached to the SWNT to allow the non-optical sequencing of DNA, cDNA or RNA molecules. Enzymes which catalyze the template-dependent incorporation of dNTPs are known to undergo well characterized conformational changes that can be used to monitor the nucleotide specific incorporation of natural or analog dNTPs or NTPs in accordance with the methods and devices described herein and thus provide the sequence of the template molecule. This label-free sequencing method has advantages over the currently practiced non-optical sequencing methods insofar as it allows the discrimination of a nucleotide specific incorporation event from a homogeneous mixture of four natural or analog dNTPs or NTPs, though the present invention is compatible with the practice of flowing individual dNTPs or analog dNTPs or NTPs in a serial and cyclic fashion for the purposes of sequence determination. The use of a Reverse Transcriptase as the non-covalently bound sensitizing molecule 30 enables the direct sequencing of RNA molecules without the need for an intermediate cDNA conversion step.

Since accuracy of correct nucleotide incorporation is of tantamount importance in DNA, RNA or cDNA sequencing, an alternative method for enhancing the detection of the specific incorporation of the correct dNTP or NTP would be to use analog dNTPs or NTPs which exacerbate the conformational dynamics of correct nucleotide incorporation thus ensuring accurate sequencing. Non-labeled analog dNTPs or NTPs which can be used to enhance the kinetic or dynamic discrimination of correct nucleotide incorporation are well known to one skilled in the art and include but are not limited to modifications of the purine and pyrimidine bases (i.e., at the C-4 and C-7 positions), the deoxyribose or ribose portions of the nucleotides, and the, alpha, beta and gamma phosphates of the dNTPs or NTPs including the use of tetra or penta-phosphates, with or without additional phosphate modifications.

Other methods of sequence accuracy enhancement that are compatible with the present invention that are known to one skilled in the art can be used including but not limited to reading the same template molecule multiple times. Other possibilities involve the use of a read twice format in which pyrophosphorolysis is used to read the same template molecule a second time.

In embodiments, a method for detecting the dynamics and kinetics of the single molecule sensing device is provided. Any method for measuring changes in electrical conductance of the SWNT can be used to monitor the single molecule sensing device. In the embodiments, a bias difference of 100 mV is applied across the SWNT, and the current flowing through the conductor is measured as a function of time using circuitry. Chemical binding or recognition at the target-specific component of the sensitizing molecule results in changes to the conductivity-modulating component of the sensitizing molecule, causing increases and decreases in the measured current. Multiple binding and unbinding events, which upon averaging comprise the chemical kinetics of the target-specific component, produce multiple current fluctuations that can be timed, counted, discriminated, analyzed or stored using signal processing techniques which are known in the art. The current fluctuations can consist of simple increases and decreases in a square-edged pattern. Alternately, the fluctuations can comprise any wavelet including shapes that are triangular, sinusoidal, or having any number of Fourier components. The amplitudes, durations, and shapes of these wavelets all encode the activity of the target-specific component and therefore can be analyzed using the computer 24 to uncover the kinetics of the binding and other mechanical and electronic degrees of freedom. Statistical analysis of these parameters provides insight into the kinetic variability, transitions, and intermediate chemical states of the target binding and unbinding processes. The degrees of freedom in the current signal distinguish among multiple similar target molecules that all bind to the same site, for example between a target molecule and an inhibitor molecule of the binding site. These degrees of freedom can also distinguish weak interactions such as molecule recognition that occur before true binding.

In embodiments, the ability to distinguish and monitor either covalent or non-covalent binding of inhibitor molecules in provided. Inhibitors of protein function are commercially important as pharmaceutical agents, including anti-viral, anti-cancer and anti-bacterial therapeutics. The testing of effective inhibitors is a time-consuming and expensive process. The device provides for directly monitoring protein function with single molecule resolution, while simultaneously probing the protein with any number of different candidate inhibitors. Using automated fluidic delivery systems well known in the art such as a flow cell, candidate inhibitor solutions can be delivered to the device one by one to identify inhibitors with the desired kinetic properties. Alternately, candidate inhibitors can be in mixtures, either as-synthesized or purposefully categorized by chemical structure or function or any other feature, in order to rapidly assay entire batches of candidate molecules.

It will therefore be seen that methods of the present disclosure are able to detect the dynamics and kinetics of a single sensitizing molecule. When the sensitizing molecule is an enzyme, the kinetics and dynamics comprise rates of enzymatic turnover or rates of conformational movements. The technical advantage of the present invention is that the dynamics and kinetics of a single sensitizing molecule can be detected, overcoming the problems of ensemble measurements that occur when multiple sensitizing molecules are present on the SWNT. Furthermore, the present disclosed methods overcome the problems associated with prior methods of fabricating single molecule devices which create a defect site on the SWNT which is then functionalized a single sensitizing molecule.

Embodiments include a method of making a single molecule sensing device. The method includes forming at least one single-walled carbon nanotube on a substrate 26 having first and second ends thereof connected, respectively, to a first electrode and a second electrode. The single-walled carbon nanotube sidewall of the device is then non-covalently functionalized with at least one functional group of at least one linker molecule containing a plurality of functional groups. A single sensitizing molecule is functionalized with at least one the functional groups of the at least one linker molecule (e.g., the functional group that is not non-covalently functionalized with the SWNT).

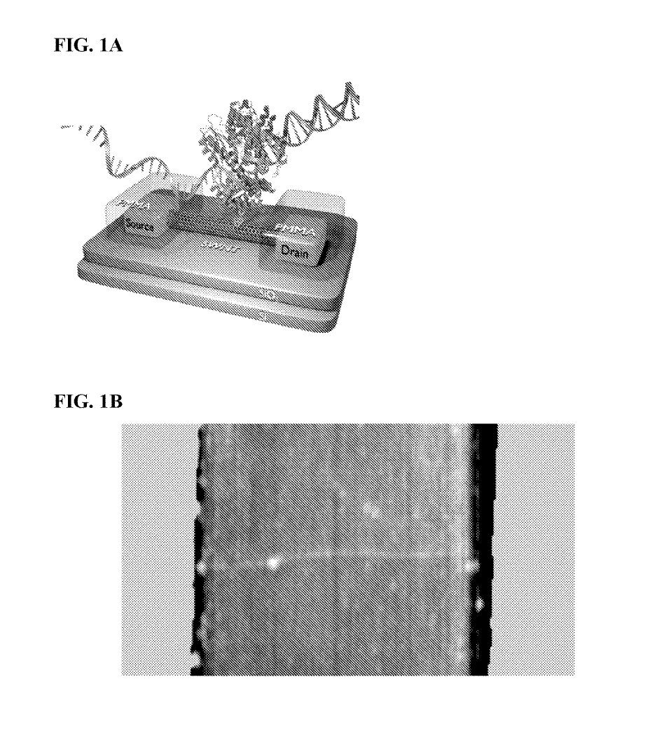

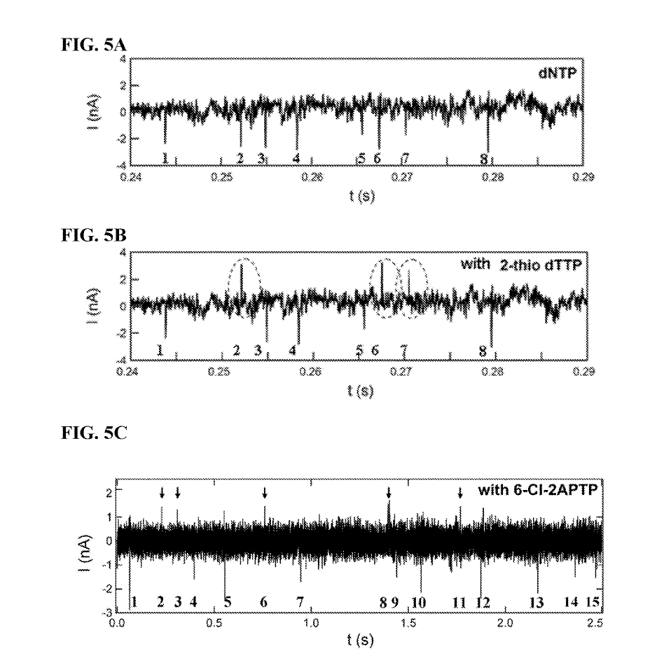

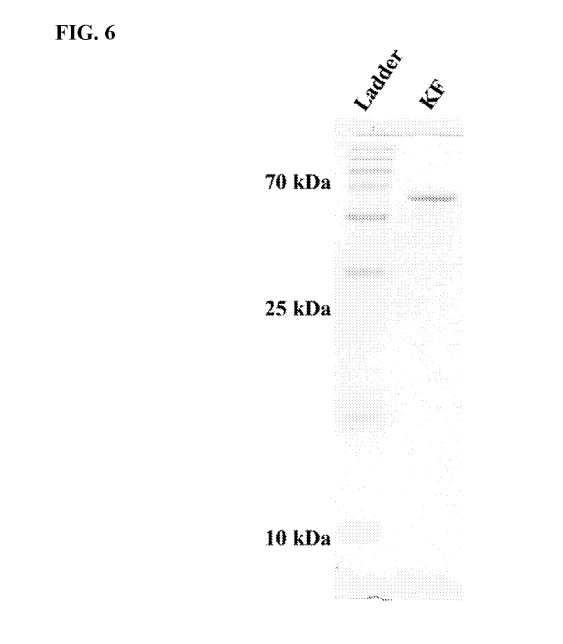

In embodiments, SWCNT-FETs are fabricated and functionalized with a single-cysteine variant of exonuclease-deficient KF (D355A/E357A/L790C/C907S). Purification of KF to >95% is ensured by its homogeneity (FIG. 6). A fluorescence-based assay confirms activity of the bulk enzyme prior to attachment (FIG. 7). Attachment of KF to SWCNT-FETs is accomplished by soaking the devices in a solution of N-(1-pyrenyl)maleimide (1 mM in ethanol, 30 min), followed by incubation with KF (300 nM KF in a standard KF activity buffer of 20 mM Tris, 50 mM NaCl, 10 mM MgCl₂, 100 μM TCEP, pH 8.0). Atomic force microscopy after data collection confirms attachment of a single KF molecule to each device (FIG. 1B). Such devices are referred to simply as KF nanocircuits.

In embodiments, homopolymeric templates poly(dA)₄₂, poly(dT)₄₂, poly(dG)₄₂, or poly(dC)₄₂ mixed with complementary dNTP analogs are used for detecting conformational change of a polymerase, e.g., DNA polymerase. In embodiments, each template is fused to an M13 priming site and mixed with an M13 forward primer in a 1:1 stoichiometric ratio; for hybridization, the mixture is heated in a thermal cycler to 95° C. for 5 to 10 min followed by cooling to 65° C. then further cooling with a gradient of 5° C. every five min until reaching room temperature. In embodiments, KF nanocircuits are immersed in activity buffer with the annealed template-primer at 100 nM concentrations. Native or analog dNTPs are added to the buffer in excess, ensuring Vₘₐₓ conditions for KF catalysis. To compensate for possibly reduced affinity of dNTP analogs, the experiments apply higher concentrations of analogs (FIG. 1C, e.g., 100 μM) than the native dNTPs (e.g., 10 μM).

In embodiments, measurements consist of monitoring the source-drain current, I(t), through the SWCNT-FET while the attached KF molecule interacted with its surrounding environment. In embodiments, the drain electrode is biased at 100 mV, and the electrolyte, which serves as a gate electrode, is held at or near 0 V. Incubation of the device with any template-primer and its complementary dNTPs transduces fluctuations, ΔI(t), whereas these fluctuations may be absent with non-complementary dNTPs or in control measurements missing the template-primer or KF attachment. In embodiments, I(t) fluctuations are amplified, digitized at 100 kHz, and stored as uninterrupted, 600 s. Between measurements, the KF nanocircuits may be rinsed twice with activity buffer, incubated in buffer for 5 min, then rinsed twice with buffer before introducing another nucleotide and template-primer. Each KF molecule may be monitored with multiple analogs, their corresponding native dNTPs, and nucleotide-free buffer in order to collect directly comparable data sets, confirm typical KF activities, and produce ΔI(t) excursions.

FIGS. 2A and 2B show representative ΔI(t) signals produced by a KF nanocircuit processing a poly(dC)₄₂ template in the presence of dGTP. In embodiments, the device produces uninterrupted sequences of negative ΔI(t) excursions, shown at three different magnifications. Each ΔI(t) excursion indicates the formation of one base pair, and the kinetic parameters derived from ΔI(t) data sets are consistent with known single-molecule analysis of KF motions and ensemble KF incorporation rates. In embodiments, G·C or C·G base pair formation may be identical to one another; A·T/T·A base pair formation also may provide very similar polymerization kinetics, dynamics and ΔI(t) values compared to each other. Measurements with the native dNTPs may provide baseline values for comparison with dNTP analogs.

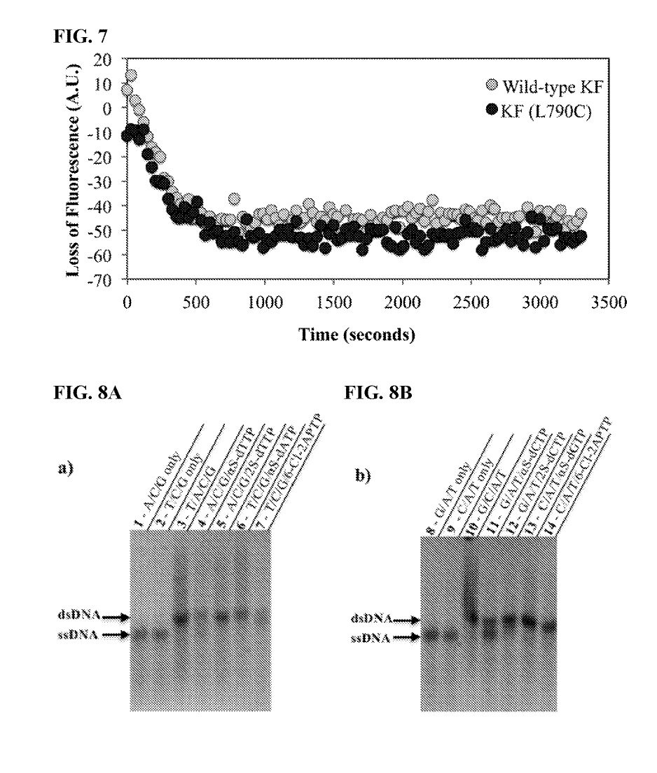

In embodiments, commercially available dNTP analogs are incorporated into DNA through KF polymerization in both ensemble and single-molecule assays (FIGS. 8A-8B). In embodiments, measurements with α-thio-dNTP, or dNTPαS, analogs produced ΔI(t) data sets may appear similar to the native dNTPs, but with different incorporation rates (FIG. 2C). In embodiments, when measured with KF nanocircuits, incorporation of 6-chloro-2-aminopurine-drTP, or 6-Cl-2-APTP, opposite both poly(dC)₄₂ and poly(dT)₄₂ templates cause ΔI(t) signals with inverted amplitude reflecting a different KF conformation (FIG. 2D). This analog incorporates more slowly; for example, opposite poly(dC)₄₂, 6-Cl-2APTP produced ΔI(t) excursions at 80% of the rate of dGTP. ΔI(t) records with the 2-thio-dNTP analogs produced mixed behaviors in which KF activity produced negative ΔI(t) excursions during one minute, positive ΔI(t) excursions during another minute, and, more rarely, mixtures of both behaviors along a single template strand (FIGS. 2E-2F).

For native dNTPs, the time constants for the experimental baseline current, τₒₚₑₙ may also be referred to as τₕᵢ. Time constants representing a native dNTP incorporation event may occur with lower current, and is referred to as τₗₒ. Positive, negative, or mixtures of both positive and negative ΔI(t) excursions are disclosed here, and time constants for either direction of excursions are termed τ_{closed}. Distributions of τₒₚₑₙ and τ_{closed} are derived from each record of polymerization data.

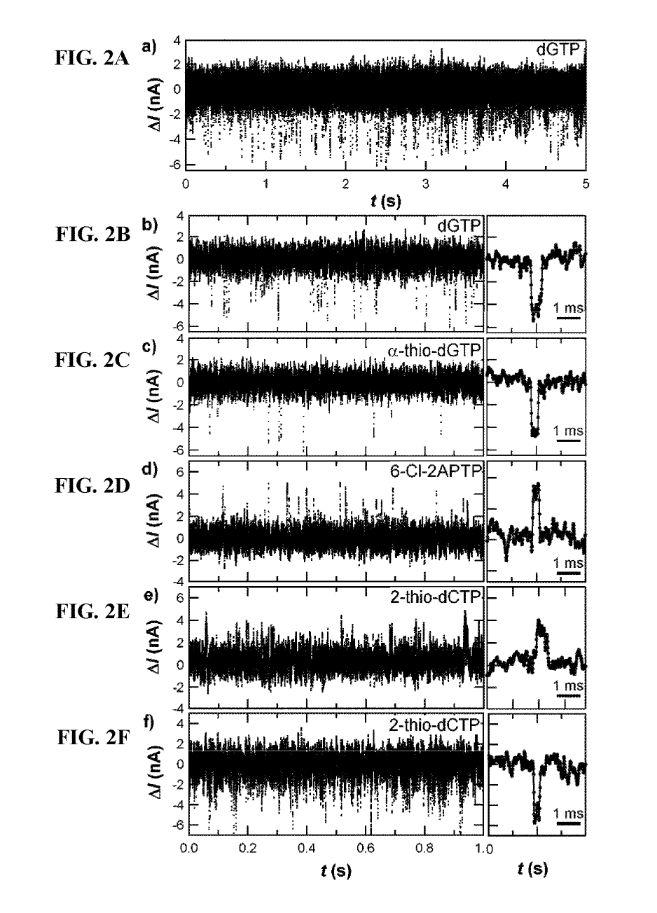

FIGS. 3A-3B show example distributions for incorporation of dGTP substrates into poly(dC)₄₂ templates. The distributions from native and analog dGTP T_{closed} events were nearly indistinguishable except for rare events in the tails, for which we have the poorest statistics (FIG. 3A). To draw comparisons between native and analog dNTPs, we focused on the mean time constant <τ> of the primary, Poissonian component of these distributions. All of the mean values for <τ_{closed}> were in close agreement around 0.3±0.1 ms. By comparison, the distributions and mean values of <τₒₚₑₙ> were clearly different. For example, KF spent 63.6±2.8 ms in its open conformation when processing α-thio-dGTP, which is 56% longer than the 40.8±0.6 ms observed for native dGTP (FIG. 3B).

The kinetic parameters <τ_{closed}>, <τₒₚₑₙ>, and the average rate of incorporation k were analyzed for the four homopolymeric templates with native and analog dNTPs (Table 1). As with the case described above, every combination produced identical τ_{closed} distributions with <τ_{closed}> values in the range of 0.3±0.1 ms. While a similar effect was previously observed for the four native dNTPs,³³ the extension of this result to dNTP analogs having different nucleobase sizes, electronic properties, hydrogen bonding, or substitution at the α-phosphodiester was unexpected.

On the other hand, Tₒₚₑₙ is more sensitive to dNTP identity. The mean duration of <τₒₚₑₙ> ranged from 23 ms with native dCTP to 145 ms with α-thio-dATP. Among the four native dNTPs, <τₒₚₑₙ> was longer for dTTP or dATP incorporation than for dGTP or dCTP incorporation. This hierarchy was preserved within longer <Tₒₚₑₙ> durations measured for all four α-thio-dNTPs. The α-thio substitution increased <Tₒₚₑₙ> by 50% in the case of dGTP and dCTP, whereas the increase was more than 100% for dTTP and dATP.

The average KF processing rate for dNTP incorporation was calculated as k=(<τₒₚₑₙ>+<τ_{closed}>)⁻¹. τₒₚₑₙ largely determines k, because it is at least 60 times longer than τ_{closed}. At its fastest, KF incorporated 2-thio-dCTP at more than 30 s⁻¹. The increase in τₒₚₑₙ described above for α-thio-dNTPs reduced k to 15 s⁻¹ for α-thio-dCTP and α-thio-dGTP and 7 s⁻¹ for α-thio-dATP and α-thio-dTTP. Rates for 6-Cl-2APTP incorporation compared most favorably to the slowest rates observed for native dGTP incorporation. Conversely, 2-thio-dTTP and 2-thio-dCTP incorporation appeared slightly faster than incorporation of their native counterparts.

Similar results were reproduced using a dozen different KF molecules. Each KF was attached to a different SWCNT-FET and measured independently. For comparison, a non-homopolymeric template measured with dNTP analogs resulted in similar kinetics (data not shown). As mentioned previously, our experiments applied 100 μM of dNTP analogs to ensure steady state conditions; for comparision, 10 μM α-thio-dATP with the poly(dT)₄₂ template did not affect DNA polymerization. Due to static disorder, some KF molecules processed faster or slower than the ensemble average, but without any significant change to the relative comparison of analog to native dNTPs.

The single-molecule experiments carried out in this study illustrate and shed new light on the well-appreciated plasticity of DNA polymerases like KF. This class of enzymes can accommodate even dramatically modified incoming dNTPs. However, we directly observe conformational motions required by the enzyme to maintain fidelity when faced with certain altered dNTPs. Reflecting the limits for such accommodations, DNA polymerases are known to exhibit strong sensitivity to minor changes in dNTP size and shape. Our analysis benefits from comparing single molecule data with native and analog dNTPs during numerous processive incorporation events. This analysis begins with the kinetics of the two observed enzyme conformations during catalysis, which were captured by τₒₚₑₙ and τ_{closed}.

Events taking place during τₒₚₑₙ include the rate-limiting step of dNTP recognition, which is sensitive to both nucleobase and backbone modifications. Successful recognition and binding of the appropriate nucleotide triggers KF's activation and closure. Previous FRET-based experiments with the related T7 DNA polymerase have identified a “fully open” conformational state resulting from mismatch recognition. However, using the L790C attachment site, the SWCNT-FET records no KF motions and no signals in the presence of mismatched dNTPs. The absence of intermediate states or mismatch-associated motions suggests that our attachment site is insensitive to this initial fidelity checkpoint. Thus, ΔI(τ) excursions result from a catalytically committed conformation, and are not restricted to simply the global motion of the enzyme opening and closing.

The dNTP analogs were chosen for their ability to be incorporated into DNA templates by DNA polymerases and variations in sizes, structures, and reactivity. We examined either substitution at the α-phosphate or nucleobase. The first type of analog, e.g., α-thio-dNTP, substituted a non-bridging, α-phosphoryl oxygen atom with sulfur to introduce a new stereocenter and alter the reactivity at this crucial site. The second category of dNTP analogs, substitution, e.g., halogen or sulfur substitution, on the nucleobase, changes the size and electronic structure of the base pair; some analogs also alter the hydrogen bonding available for base pairing. For example, 6-Cl-2-APTP (FIG. 1C), has two hydrogen bonding profiles, allowing its incorporation opposite both T and C bases. Compared to dATP, 6-Cl-2-APTP replaces the 6-amino group with chlorine, but introduces a 2-amino functionality; this configuration ultimately provides the same number of Watson-Crick hydrogen bonds complementary to T as dATP. When used as a dGTP analog, 6-Cl-2-APTP has different tautomerization, which changes the N−1 from a hydrogen bond donor to an acceptor. In this case, replacement of oxygen with chlorine dramatically decreases the strength of the hydrogen bonding.⁴¹ Like 6-Cl-2APTP, sulfur-substituted analogs 2-thio-dTTP and 2-thio-dCTP also form larger base pairs due to the increased bond length of the thiocarbonyl.

In embodiments, the dNTP or NTP analog includes a chemical modification at the triphosphate moiety. In embodiments, the chemical modification at the triphosphate moiety is substitution of an O at the α-position with S. In embodiments, the triphosphate moiety of any dNTP or NTP analog has the structure of Formula (I),

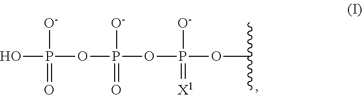

wherein X¹ is S or O. In embodiments, the dNTP analog is α-thio-dATP, α-thio-dGTP, α-thio-dCTP, or α-thio-dTTP. In embodiments, the NTP analog is α-thio-ATP, α-thio-GTP, α-thio-CTP, or α-thio-TTP. In embodiments, a dNTP or NTP analog includes a substitution at the α-position of the triphosphate moiety as set forth in Formula (I), and one or more substitutions at the nucleobase as disclosed herein and known in the art.

In embodiments, the dNTP or NTP analog is substituted at the nucleobase. In embodiments, a purine is substituted as shown in Formula (IIa),

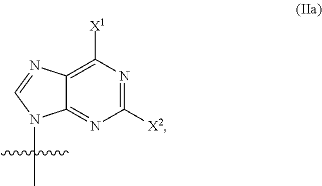

wherein X¹ is hydrogen, halogen or —NH₂, and X² is hydrogen or —NH₂. In embodiments, X¹ is —NH₂, and X² is hydrogen, providing dATP or ATP. In embodiments, X¹ is not —NH₂, and X² is —NH₂, providing 6-substituted 2-APTP or substituted analog thereof. In embodiments, the analog is 6-Cl-2-APTP, also known as 6-Cl-dGTP.

In embodiments, the dNTP or NTP analog includes a nucleobase which is 8-oxoguanine, 2,6-diamino-4-oxo-5-formamidopyrimidine, N⁶-methyl-adenosine, O⁶-methylguanosine, N²-methyl-guanosine, 2,6-diaminopurine, indolyl, 5-methylindolyl, 5-alkyl-indolyl (e.g., 5-ethyl-indolyl), 5-ethylene-indolyl, 5-nitro-indolyl, 4-nitro-indolyl, 5-phenyl-indolyl, 5-halo-indolyl (e.g., 5-F-indolyl), 5-amino-indolyl, or 6-nitro-indolyl.

In embodiments, the dNTP or NTP includes a nucleobase with structure of Formula (IIb),

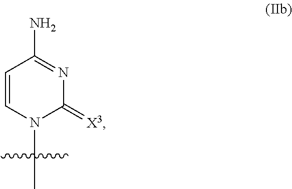

wherein X³ is O or S. In embodiments, X³ is O, providing a cytosine nucleobase. In embodiments, X³ is S, providing a 2-thio nucleobase. In embodiments, the nucleobase is 2-thio-dCTP or 2-thio-CTP.

In embodiments, the dNTP or NTP includes a nucleobase with structure of Formula (IIc),

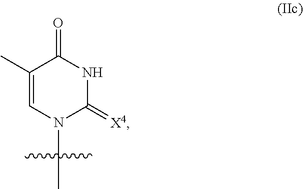

wherein X⁴ is O or S. In embodiments, X⁴ is O, providing a thymine nucleobase. In embodiments, X⁴ is S, providing a 2-thio nucleobase. In embodiments, the nucleobase is 2-thio-dTTP or 2-thio-TTP.

In embodiments, the dNTP or NTP includes a nucleobase with structure of Formula (IId),

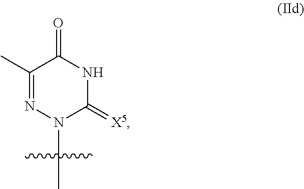

wherein X⁵ is O or S. In embodiments, X⁵ is O, providing a 6-aza-thymine nucleobase. In embodiments, X⁵ is S, providing a 6-aza-2-thio nucleobase. In embodiments, the nucleobase is 6-aza-2-thio-dTTP or 6-aza-2-thio-TTP.

In embodiments, the analogue is alpha-thio dATP, dTTP, dCTP, and dGTP, 2-thio dATP, dTTP, dCTP, and dGTP, 2-amino-6-Cl-purine-2′-deoxyriboside-triphosphate (called 6-Cl dGTP or 6-Cl-2APTP), 4-thio dTTP, 2-aza dTTP, 5-fluoro dTTP, or gamma-ANS dTTP.

In embodiments, the differences observed in τₒₚₑₙ largely reflect the mechanisms for recognizing and binding unnatural dNTPs. Long tails in the distributions for α-thio-dGTP and 6-Cl-2APTP compared to native dGTP may have been responsible for the <τₒₚₑₙ> increase (FIG. 3B). The tails can be fit to second exponentials with time constants of 200 ms, about five times longer than <Tₒₚₑₙ> for native dGTP. Similar long tails may be observed with all dNTP analogs disclosed herein, illustrating the challenges faced by the enzyme when incorporating non-natural substrates. Steps other than recognition potentially take place during the τₒₚₑₙ reported here; covalent bond formation is one possible example that would occur too quickly, even with the slowed reactivity of α-thio-dNTPs, to be detectable as rate-limiting. Faster rates of incorporation observed with the 2-thio analogs can result from more stable base pair formation, effectively shortening <τₒₚₑₙ> values. The larger size of the 2-thio-dCTP sulfur atom at the hydrogen bonding interface with the template G base does not appear to affect the ability of 2-thio-dCTP to base pair efficiently. In embodiments, the method of detecting a nucleic acid polymerase conformation detects an increase in polymerization efficiency with 2-thio-dTTP and 4-thio-dTTP compared to dTTP incorporation.

The 6-Cl-2APTP analog, with much weaker hydrogen bonding and consequent imperfect base pairing compared to dGTP, exemplifies the challenges of base pairing recognition during KF-catalyzed DNA polymerization. Longer <Tₒₚₑₙ> values for 6-Cl-2APTP versus dGTP incorporation opposite a poly(dC)₄₂ template illustrate the willingness of DNA polymerases to accept unnatural dNTPs in part by lengthening the time allotted for recognition. The <τₒₚₑₙ> value, and thus the rate of incorporation, observed during 6-Cl-2APTP polymerization opposite poly(dC) fell between the values measured for native dGTP and dATP incorporation opposite complementary, homopolymeric templates. Thus, despite its altered tautomerization and consequent loss of at least one base pairing hydrogen bond when compared to dGTP, 6-Cl-2APTP can still be incorporated more quickly than native dATP. In embodiments, the method of detecting a nucleic acid polymerase conformation detects that the base pairing hydrogen bond in the minor groove remains unchanged when 6-Cl-2APTP is considered a dGTP analog, and could govern the relatively faster rates observed for dGTP, dCTP, and 6-Cl-2APTP opposite a poly(dC) template.

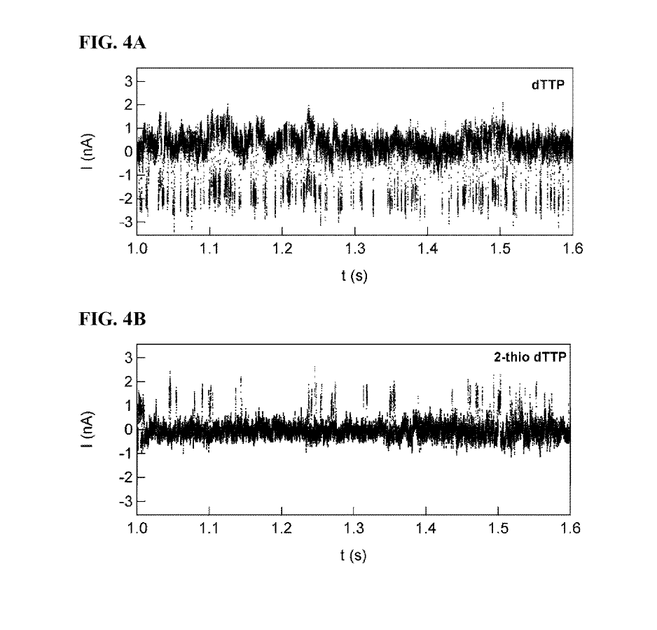

In embodiments, see, e.g., FIG. 5A, KF processes heterogeneous substrates in the presence of all four natural nucleotides (dNTP), each base pair incorporation produces a negative current spike ΔI<O. Individual spikes can be enumerated as shown in FIG. 5A, but in general they do not differentiate one type of base from another.

In embodiments, see, e.g., FIG. 5B, 2-thio-dTTP analog is used. With the thiolated deoxythymidine, positive spikes indicate (#2, 6, 7) the locations where T nucleotides were incorporated. This observation can be extended to RNA sequencing by replacing KF by an RNA polymerase. The process depicted in FIG. 5B identifies all of the T nucleotide incorporations in a particular DNA substrate. The process can be extended to all four bases by measuring the substrate with each of the four different thiolated nucleotides.

In embodiments, see, e.g., FIG. 5C, when KF processes heterogeneous substrates in the presence of natural nucleotides (dNTP) mixed with certain analogs, the resulting pattern contains positive and negative current spikes that can be used to identify a chosen base. In embodiments, three native nucleotides (dATP, dTTP, dCTP) mixed with 6-Cl-2APTP as an analog for G incorporations, are used. Current spikes in this embodiment are numbered to enumerate 15 base incorporation events. Most of the events (#1, 4, 7, 9, 10, 13, 14, 15) may consist of a single negative current spike that identifies incorporation of a native nucleotide. In embodiments, five of the events (e.g., #2, 3, 6, 8, 11; highlighted with arrows in FIG. 5C) are positive current spikes that identify 6-Cl-2APTP nucleotide incorporations. In embodiments, when 6-Cl-2APTP is used as an analog of the native dGTP nucleotide, the events (e.g., #2, 3, 6, 8, 11; highlighted with arrows in FIG. 5C) identify G nucleotides in the DNA sequence.

In embodiments, two of the events (#5, 12) shown in FIG. 5C contain a pair of closely-spaced current spikes. These pairs may indicate incorporation of one native and one 6-Cl-2APTP nucleotide in rapid succession. Alternately, a pair of spikes could be a unique signal resulting from 6-Cl-dAPTP acting as a pseudo-analog of dATP.

In embodiments, the method of detecting conformation change in a polymerase is used to sequence an oligonucleotide. The information obtained (e.g., FIGS. 5A-5C) informs the sequence of an oligonucleotides, as the spikes indicate which nucleotide or analog is being incorporated as the polymerase moves along its substrate.

In addition to recognition and binding, prolonged <τₒₚₑₙ> values for α-thio-dNTP incorporation could result from the reduced stability of the newly synthesized DNA. KF-catalyzed processing of homopolymeric templates can result in distorted dsDNA. Furthermore, α-thio-dNTPs are particularly prone to form less stable binary complexes with unfavorable DNA backbone interactions, which progressively slow the catalytic rate of KF. More pronounced effects on this step, compared to experiments with the respective native dNTPs, were observed during α-thio-dATP/α-thio-dTTP versus α-thio-dGTP/α-thio-dCTP incorporation. In embodiments, the method of detecting a nucleic acid polymerase conformation indicates sequence-dependent DNA instability, which underscores a caveat when homopolymeric templates are used. Alternatively, this difference could suggest that the α-thio substitution further interferes with the mechanism that causes <τₒₚₑₙ> to be longer for native A·T/T·A base pairs. Some of the variation in τₒₚₑₙ associated with α-thio-dNTP incorporation could result from the weakly inhibitory Rₚ stereoisomer (Kᵢ≈30 μM), present at an approximately 1:1 ratio with the Sₚ stereoisomer in the commercial synthesis of this analog. This inhibition is about an order of magnitude weaker than the Kₘ for the native dNTP,¹⁰ and thus can be expected to affect <τₒₚₑₙ> values only modestly.

During τ_{closed}, KF undergoes a distinct conformational change corresponding to formation of one phosphodiester bond between the incoming nucleotide and the nascent dsDNA. In substrate-limited experiments, the number of ΔI(t) excursions matched the number of overhanging template bases; thus, the conformational change during τ_{closed} must occur for each successful, processive nucleotide incorporation. Earlier, the short and equal duration of <τ_{closed}> for native dNTPs supported a model in which τ_{closed} results from the covalent bond-forming step itself.³³ In embodiments, the method of detecting a nucleic acid polymerase conformation uses three observations with dNTP analogs. First, the direction of ΔI(t) excursions is reversed for some dNTP analogs. Second, incorporation of 2-thio-dNTP analogs produces mixtures of both positive and negative ΔI(t) excursions. Third, as shown in Table 1, the invariance in <τ_{closed}> extended to all analogs tested despite substitutions at the electrophilic α-phosphate or the likely alternative conformations needed to accommodate substitutions on the nucleobase.

In this electronic technique, the underlying SWCNT-FET is extremely sensitive to electrostatic gating by the protein's charged surface residues within 1 nm of the attachment site. Different variants of the same enzyme can exhibit either positive or negative ΔI(t) excursions depending on the charge of the SWCNT-adjacent residues and the directions of their motion. In embodiments, during the method of detecting a nucleic acid polymerase conformation, KF and its charged residues electrostatically gating the SWCNT-FET may remain invariant. Variable ΔI(t) excursions may indicate that the residues adjacent to the KF attachment site are adopting different motions in response to certain dNTP analogs during a catalytically competent cycle. Such motions may be transmitted from the KF active site through allostery, but they may not be the motions of covalent bond formation. In embodiments, the covalent step may not proceed by the same mechanism and with the same <τ_{closed}> duration but with two opposing motions. Instead, the relevant residue motions responsible for τ_{closed} may be independent of both initial molecular recognition and the chemical step of KF catalysis.

In embodiments, in the method of detecting a nucleic acid polymerase conformation, KF is attached to the SWCNT-FET through the protein's L790C sidechain in the “fingers” subdomain, linking the electrostatic gating motions of relevant charged residues to catalytically committed motions during τ_{closed}. Each τ_{closed} event may result from the active site O-helix itself or a particular O-helix residue twisting in two possible directions during the observed stage of successful nucleotide incorporation. This proposed twisting is inferred by considering active site residue motions during known stages of nucleotide incorporation and their effect on the theoretical proximity of charged residues to the SWCNT-FET. For example, smFRET experiments with KF reveal an intermediate conformation of the active site O-helix between the open and closed states; a potentially analogous “ajar” conformation is observed in the crystal structures of the KF homolog Bst Pol I. The C-terminus of the Bst Pol I O-helix kinks on the pathway to closure such that a large shift of the KF Y766 equivalent is accompanied by a subtle rotation of the KF F762 equivalent. The rotation of the KF F762 equivalent continues until enzyme closure.

By comparing crystal structures of KF and Bst Pol I, charged residues adjacent to the SWCNT-FET are identified that could move in response to rotations by Y766 and F762 in the KF active site. In embodiments, in the method of detecting a nucleic acid polymerase conformation, the source of ΔI(t) excursions is additional motions of Y766 and/or F762 after enzyme closure and base incorporation that continue to propagate to charged residues near the SWCNT-FET. An additional KF conformational change when the nascent base pair moves to the KF post-insertion site has been observed by smFRET following successful nucleotide incorporation, and is possibly the motion measured by τ_{closed}. Significant interactions imparted by aromatic active site residues could include π-π stacking with the newly formed base pair. Such a motion would assess the electronic configuration of the base pair and interrogate the fidelity of the bond formation step without requiring hydrophilic interactions, which are altered by the dNTP analog's substitutions.

DNA polymerases, including the ones disclosed, are highly amenable to mutagenesis to tailor their properties, including for DNA sequencing applications. In embodiments, the method of the present disclosure involves mutations introduced into the DNA polymerase for bioconjugation of the enzyme to the carbon nanotube. In embodiments, such mutations alter the properties of the DNA polymerase for more efficient incorporation of unnatural bases and altering the processivity of the enzyme. In embodiments, such mutagenesis is used to improve the electrical readout and properties of the enzyme. For example, in embodiments, mutations are introduced into the enzyme active site to accommodate specific dNTP analogs with shapes or functionalities complementary to mutations made to the enzyme active site. In embodiments, the DNA polymerase is mutated to enhance the electrical response resulting from each base incorporated during DNA polymerization; for example, the substitution of an enzyme's charged residues close to the carbon nanotube provided rationalizable responses during enzyme motions.

The KF nanocircuit reveals larger ΔI(t) excursions for the A·T/T·A set than the G·C/C·G set of base pairs. Structural results have suggested that A and T template bases are most deeply buried in the DNA polymerase active site, and, therefore, the swiveling of the O-helix could be maximized. KF E710 and Y766 and other homologous active site glutamate and tyrosine residues have been implicated in a mechanism for stabilization of A·T/T·A base pairs over G·C/C·G base pairs. In embodiments, the hydrogen bonding interaction between KF E710 and KF Y766 prior to nucleotide incorporation could influence the size and shape of the active site and may play an important role in the τ_{closed} step of dNTP analog recognition.

In embodiments, similar results during incorporation of 2-thio-dTTP and 2-thio-dCTP illustrate KF's preferential recognition of the base pair's electronic structure are observed. Although the sulfur substitution only affects a Watson-Crick hydrogen bond acceptor in the 2-thio-dCTP analog, both 2-thio-dNTP analogs result in mixtures of positive and negative ΔI(t) excursions and thus both cause similar KF motions during incorporation. The sulfur substitution for the 2-thio-dNTP analogs is minor compared to the more dramatic electronic variations introduced into 6-Cl-2APTP, but the enzyme responds in a similar, although non-exclusive, manner. The observed mixtures of both negative and positive ΔI(t) excursions suggest that KF accesses both native and alternative motions, respectively, during incorporation of the 2-thio-substituted dNTPs. An apparent memory effect locks the enzyme into one motion or the other for tens of seconds, implicating an additional conformational change that is energetically bistable in the special case of 2-thio-dNTPs.

In embodiments, the method of detecting a nucleic acid polymerase conformation includes a shuttling of the nascent DNA to the inactive exonuclease (exo) domain as a possible source of positive ΔI(t) excursions. Upon melting of an unstable primer terminus due to imperfect base pairing, DNA shuttles to and from an inactive exo domain, and KF undergoes distinct conformational changes. However, such transitions occur distant from the attachment site and positive ΔI(t) excursions observed here do not change durations of <τ_{closed}>. Accordingly, shuttling to the exo domain seems inconsistent with the observation of positive ΔI(t) excursions. Similar to the conformational steps known to occur during mismatched dNTP recognition, shuttling to the exo domain must take place during τₒₚₑₙ. The ΔI(t) excursions of the present disclosure may occur during a committed catalytic cycle, and may represent an adaptable KF motion consistent with a swiveling O-helix testing the electronic integrity of the newly formed DNA base pair.

In embodiments, the method of detecting a nucleic acid polymerase conformation, dNTP analogs challenge the limits of nucleotide incorporation by DNA polymerases, including the stereochemistry at the electrophilic phosphate, the hydrogen bonding capability of the incoming base, and the mechanisms of fidelity checking. Since most dNTP analogs increase average <τₒₚₑₙ> and the broadness of its kinetic distributions, the rate-determining dNTP recognition step appears highly sensitive to even minor variation in substrate structure. However, dramatic substitutions at the reactive site of bond formation fail to impact the durations of <τ_{closed}>. The direction of the ΔI(t) excursions, on the other hand, switches to positive or a mixture of both negative and positive signals with base-modified dNTP analogs. Since these dNTP analogs have functionalities at the bond formation center identical to native substrates, the dramatic changes in ΔI(t) direction may result from fidelity checking by KF before opening to process the next substrate. Such events can be readily distinguished from native dNTP incorporation events and provide direct observation of the enzyme accommodating unnatural dNTPs by reversing the direction of its dynamic error checking.

### EXAMPLES

### Example 1. Incorporation of Deoxynucleoside Triphosphate Analogs by Single-Molecule DNA Polymerase I (Klenow Fragment) Nanocircuits

Introduction.

To ensure survival of all known life forms, DNA polymerases must correctly recognize incoming deoxynucleoside triphosphate (dNTP) substrates and successfully catalyze their incorporation into new strands of DNA. The required fidelity of all DNA polymerases relies partially on Watson-Crick basepair complementarity of incoming dNTPs hybridizing to a single-stranded DNA template. [1, 2] However, unnatural dNTPs incapable of hydrogen-bonding with native, complementary bases have also been successfully incorporated by DNA polymerases. Such analogs can form stable base pairs with a shape similar to the canonical A·T and G·C base pairs. [3-5] Studies with dNTP analogs have uncovered requirements for stereochemistry, geometry, electronic effects and hydrophobic interactions during nucleotide incorporation. [6-9]

Results from dNTP analog incorporation experiments clarify nucleotide selection criteria by DNA polymerases, including requirements for further chain extension and efficient catalysis. For example, catalytic rates from polymerization with phosphorothioate analogs determined the stereochemical preference for nucleophilic attack by the priming 3′-OH onto the electrophilic α-phosphate of the dNTP. [6] Small modifications of the nucleobases, including thio- and halo-substitution in hydrogen bonding positions, result in altered incorporation rates and demonstrate the requirement for tight steric fit in the DNA polymerase active site. [8,10]] The Watson-Crick-like geometry required to incorporate unnatural bases is induced by several interactions between the polymerase active site and emerging base pair. [5]

Detailed evaluation of unnatural dNTP polymerization beyond a single base pair could provide accurate kinetic information about this non-native polymerase activity. Furthermore, translation of associated small conformational changes during base recognition into a reliable measurement could reveal new aspects of the roles for sterics and electronics in DNA polymerization. As reported here, such information can be elucidated by single-molecule techniques.

Conventional studies of bulk or ensemble populations of enzymes cannot observe intermediate steps and transition states in a reaction mechanism. However, experiments with individual molecules allow observation of such states that would otherwise be averaged in an ensemble population.[11-13] DNA polymerization experiments employing single-molecule Förster resonance energy transfer (smFRET) have revealed conformational flexibility and insights into the fidelity mechanism of DNA Polymerase I Klenow Fragment, termed KF hereafter.[14-16] Despite its power to capture new information about enzyme dynamics, smFRET requires a fluorescently labeled protein and/or substrate. Photobleaching and the flux of photons between fluorophores limit both the duration and time resolution, respectively, of smFRET experiments.

Recently, we have described a new approach to single molecule enzymology and applied it to three enzymes. In this technique, an individual protein is bioconjugated to a single walled carbon nanotube field effect transistor (SWCNT-FET; FIG. 1A). The approach uncovered new insights into the number of steps, kinetic parameters and processivity of T4 lysozyme, an enzyme studied for over 100 years.[17,18] The tremendously dynamic rates of Polynucleotide Kinase A (PKA) uncovered the enzyme's role as a highly regulatable molecular switch.[19] In examining KF conjugated to SWCNT-FETs, significant differences between A·T/T·A and G·C/C·G base pairing demonstrated that the enzyme's closed conformation depends upon the identity of the incoming dNTP.[20] The sensitivity to this difference was surprising since the Watson-Crick base pairs have similar sizes, and it indicated that the SWCNT-FET technique might also be responsive to the unique kinetics and conformations associated with dNTP analogs.

Here, the SWCNT-FET technique was used to distinguish the differences between native dNTPs and dNTP analogs during incorporation by KF. Using thio- and halo-substituted dNTP analogs, DNA polymerization was monitored and then statistically analyzed to reveal differences in incorporation kinetics for some, but not all, dNTP analogs. Furthermore, the time each analog spent in the enzyme open and closed conformations uncovers variations in times required for ratelimiting steps. The results provide a portrait of an enzyme grappling with challenges in molecular recognition during catalysis.

Experimental.

SWCNT FETs were fabricated [17] and functionalized with a single cysteine variant of exonuclease-deficient KF. Purification of KF to >95% ensured its homogeneity. A fluorescence-based assay confirmed activity of the bulk enzyme prior to attachment. [20,21] Atomic force microscopy confirmed attachment of individual KF molecules (FIG. 1B) after data collection from each device.

For each measurement, dNTPs were chosen to complement the homopolymeric templates poly(dA)₄₂, poly(dT)₄₂, poly(dG)₄₂, and poly(dC)₄₂. Each template was fused to an M13 priming site and mixed with an M13 forward primer in a 1:1 stoichiometric ratio; for hybridization, the mixture was heated to 95° C. for 5 to 10 minutes followed by cooling to room temperature. SWCNT FETs were immersed in a standard DNA Pol I activity buffer (20 mM Tris, 50 mM NaCl, 10 mM MgCl₂, 100 μM TCEP, pH 8.0) with the template-primer hybrid at 100 nM concentration. Native or analog dNTPs were added to the buffer in excess, ensuring Vₘₐₓ conditions for KF. To compensate for the slower incorporation of dNTP analogs, the experiments applied higher concentration of analogs (FIG. 1C, 100 μM, Trilink Biotechnologies) than the native dNTPs (10 μM, Fisher).

Measurements consisted of monitoring the source-drain current I(t) of a SWCNT FET while the attached KF molecule interacted with its surrounding environment. The FET was biased at 100 mV, and the electrolyte, which served as a gate electrode, was held at 0 V. Incubation of the device with any nucleotide and its complementary template-primer transduced fluctuations ΔI(t) that were measured with a current preamplifier (Keithley 428), digitized at 100 kHz, and stored for later analysis. Between measurements, the KF nanocircuits were rinsed twice with assay buffer, incubated in buffer for 5 minutes, then rinsed twice again with buffer before introducing another nucleotide. Each KF molecule was monitored with multiple analogs, the corresponding natural dNTPs, and nucleotide-free buffer in order to collect directly comparable data sets, confirm typical KF activities, and reproduce the types of ΔI(t) reported previously.[20]

Results.

Incubation of the nanocircuit with a native dNTP and its complementary template-primer caused negative changes in current, ΔI(t) (FIG. 2A). As reported previously, rapid current fluctuations only occurred in the presence of KF, dNTP and template-primer. Each ΔI(t) excursion correlates with the closure of the KF enzyme. Thus, the kinetic parameters derived from measurements of such current excursions with native dNTPs were consistent with previous analysis of enzyme motions (e.g., FIG. 2A). [20] These measurements provided baseline values for comparison with the dNTP analogs (Table 1).

TABLE 1
Kinetics of native and analog dNTP incorporation by KF.^{a}

| Template | Nucleotide | <τₒₚₑₙ (ms)> | <τ_{closed} (ms)> | k (1/s) |
|---|---|---|---|---|
| poly(dT)₄₂ | dATP | 58.9 ± 1.2 | 0.34 ± 0.18 | 16.8 ± 0.4 |
| (SEQ ID | α-thio-dATP | 145.9 ± 8.4 | 0.38 ± 0.21 | 6.8 ± 0.4 |
| NO: 9) | | | | |
| poly(dA)₄₂ | dTTP | 69.6 ± 2.3 | 0.33 ± 0.12 | 14.3 ± 0.5 |
| (SEQ ID | α-thio-dTTP | 152.1 ± 6.6 | 0.29 ± 0.13 | 6.6 ± 0.3 |
| NO: 10) | 2-thio-dTTP | 61.1 ± 3.2^{b} | 0.23 ± 0.14^{b} | 16.3 ± 0.9^{b} |
| poly(dG)₄₂ | dCTP | 42.8 ± 5.0 | 0.35 ± 0.20 | 23.2 ± 3.2 |
| (SEQ ID | α-thio-dCTP | 68.8 ± 4.6 | 0.33 ± 0.19 | 14.5 ± 1.0 |
| NO: 11) | 2-thio-dCTP | 32.3 ± 1.1^{b} | 0.41 ± 0.15^{b} | 30.6 ± 1.2^{b} |
| poly(dC)₄₂ | dGTP | 40.8 ± 6.0 | 0.40 ± 0.20 | 24.3 ± 4.3 |
| (SEQ ID | α-thio-dGTP | 63.6 ± 2.8 | 0.21 ± 0.15 | 15.7 ± 0.7 |
| NO: 12) | 6-Cl-2APTP | 50.5 ± 1.4 | 0.20 ± 0.12 | 19.7 ± 0.6 |

^{a}Average values ± standard deviation
^{b}Similar values were observed for both up- or down-switching events.

The dNTP analogs examined either substitution at the α-phosphate or alteration to the nucleobase. In the first category, substitution of a phosphoryl oxygen atom with sulfur introduces a new stereocenter into the dNTP to create α-thio-dNTP, also known as dNTPαS. Commercially synthesized without control over stereochemistry, the diastereomeric ratios around this α-phosphorus were likely 1:1. In the second category of dNTP analogs, halogen or sulfur substitution on the nucleobase could introduce a larger base with altered tautomerization and consequently different hydrogen bonding. For example, the analog 6-chloro-dGTP (also known as 6-Cl-2-APTP) has different tautomerization than dGTP, which changes N−1 from a hydrogen bond donor to an acceptor. Also, replacement of oxygen with chloride decreases the strength of hydrogen bonding. Other examples of modified nucleobases, 2-thio-dTTP and 2-thio-dCTP, replace oxygen with sulfur to increase the size of these bases. Altered kinetics resulting from incorporation of these analogs were expected to result in distinctive electronic signals, perhaps slowing the enzyme recognition of the incoming dNTP.

Phosphorothioate analogs (α-thio-dNTPs), 6-Cl-dGTP, 2-thio-dTTP, and 2-thio-dCTP were incubated with complementary template-primers. As observed with the native dNTPs, negative current fluctuations resulted during α-thio-dNTP incorporation (FIG. 2B). Conversely, 6-Cl-dGTP incorporation caused positive current fluctuations (FIG. 2C). The analogs containing 2-thio substitutions caused a mixture of both negative and positive current fluctuations. Both α-thio-dGTP and 6-Cl-dGTP analogs produced ΔI(t) excursions two to three times less frequently than dGTP.

Distributions for τₒₚₑₙ and τ_{closed} were derived from more than 50 s of polymerization data with dGTP and its two analogs (FIGS. 3A-3B). The data fit simple Poisson distributions with single <τ> time constants. Fits of distributions for native and analog dGTP τ_{closed} events only slightly deviate from each other, especially at the tails. Most events overlapped with high probability (˜50%). The mean duration of closed complexes in the presence of dGTP, α-thio-dGTP or 6-Cl-dGTP were in close agreement (0.2-0.4 ms). For native dGTP, a single exponential fit to the data encompassed >90% of τₒₚₑₙ events. However, an analogous fit to the α-thio-dGTP or 6-CldGTP data could only encompass ≈75% of τₒₚₑₙ events. In the KF open conformation, only 20 to 30% of the analog events overlap with the exact time constant required for native dGTP. The kinetics of incorporation with dGTP analogs deviate significantly from the native, and their average time constant could be readily distinguished.

This analysis of kinetic parameters τ_{closed}, τₒₚₑₙ, and the rate of incorporation (k) was extended to the other native and analog substrates (Table 1). As described for α-thio-dGTP and 6-Cl-dGTP, formation of the phosphodiester bond during the KF closure, quantified by τ_{closed}, lasts between 0.2 and 0.4 ms for all dNTPs examined, including native and analogs. The mean duration of the KF open conformation, τₒₚₑₙ, for all α-thiodNTPs was 2 to 3 times longer than for their native dNTPs. For example, KF spends approximately 2.5 times longer in the open conformation, τₒₚₑₙ, when processing α-thio-dGTP (63±3 ms) than with native dGTP (24±1 ms). As has been reported for native dNTPs,[20] α-thio-dCTP and α-thio-dGTP incorporated faster into the nascent strand than α-thio-dATP and α-thio-dTTP. The τₒₚₑₙ-dominating A·T/T·A base pair formation is 2 to 3 fold longer than τₒₚₑₙ for G·C/C·G base pairs.

The τₒₚₑₙ and τ_{closed} values reveal the times required for full cycles of dNTP incorporation. The average KF processing rate was calculated as k=1/(<τₒₚₑₙ>+<τ_{closed}>). Given the much greater amount of time spent in the open conformation (>98%), the duration of τₒₚₑₙ largely determines the rate of enzymatic catalysis. Average KF processing rates decreased 2 to 3 fold for the α-thio-dNTP and 6-Cl-dGTP analogs, as τₒₚₑₙ was 2 to 3 times longer. For example, time spent in the KF open conformation when processing 6-Cl-dGTP (<τₒₚₑₙ>=50±1 ms) is about twice as long as with dGTP processing (<τₒₚₑₙ>=24±1 ms), resulting in a rate of 20 s₋₁ compared to 41 s₋₁, respectively. Rates for 2-thio-dTTP (<k>=16 s₋₁) and 2-thio-dCTP (<k>=36 s₋₁) were approximately the same as their native counterparts.

Discussion.

The phosphodiester bond formation appears indifferent to the analog substitutions, even with the α-phosphate modified. The sulfur of α-thio-dNTP replaces a non-bridging oxygen atom in a key electrophilic functionality. Despite the weakened electrophilicity of the phosphate and consequent decreased reactivity of the thiophosphodiester versus the phosphodiester, the bond exchange step is still rapid, and not ratelimiting. [22,23]

Approximately twenty-five percent of α-thio-dGTP and 6-Cl-dGTP τₒₚₑₙ events deviated from the single exponential fit to the data, which is consistent with greater outlier events taking place as the enzyme struggles to close around the unnatural dNTP. The slower time constants for τₒₚₑₙ confirms the longer time required for the rate-limiting, incoming dNTP recognition step inherent to a modified base. However, substantial overlap between the distributions for native and analog dGTP prevents instantaneous assignment of the KF conformation for each event.

A previous study determined the Sₚ diastereomer of α-thiodNTP as the solely preferred substrate for DNA Pol I incorporation.₆ The observed rates for α-thio-dNTP processing could stem from the pseudo-substrate inhibition of DNA polymerase by the Rₚ diastereomer. The Rₚ diastereomer of α-thio-dTTP binds weakly to the KF active site, and inhibits its catalysis with Kᵢ≈30 μM. This inhibition constant is about an order of magnitude weaker than the Kₘ for the native dNTP.₆ Though the non-stereochemically controlled α-thio-substitution effectively removes half of the available substrate, a large excess of α-thio-dGTP ensured such effects were unlikely to be the cause of the dramatic lengthening of time spent in the enzyme's open conformation. In addition, the diastereomeric mixtures of α-thio-dNTPs could also affect the stability of the DNA backbone of the synthesized strand. [24,25] However, formation of the phosphodiester backbone, which takes place during the enzyme's closed conformation, quantified by τ_{closed}, remains unchanged. Therefore, the most likely culprit in slowing the kinetics of α-thio-dNTP is inhibition by the Rₚ stereoisomer during the incoming base recognition step. Thus, KF finds dNTPs with the correct stereochemical configuration, rejecting incorrect substrates competing to bind and inhibit the enzyme.

Previous studies have shown efficient catalysis by DNA polymerase a to incorporate 6-Cl-dGTP in base-pairing to a poly(dC) template. [26]] Though 6-Cl-dGTP can be incorporated opposite the base T by T4 DNA polymerase,[27,28] this capability has not been explored to date in a KF functionalized nanocircuit. The experiments with 6-Cl-dGTP described here illustrate the willingness of DNA polymerases to accept unnatural dNTPs with incorrect Watson-Crick base pairing. In order to incorporate these non-native bases successfully, DNA polymerase must apply alternatives to conventional hydrogen bonding recognition criteria for nucleotide selection. The kinetic results are consistent with the adaptation of a rate limiting step associated with hydrogen bonding during dNTP recognition.

It was hypothesized that conformational changes following discrimination of larger-sized bases 6-Cl-dGTP, 2-thio-dTTP, and 2-thio-dCTP could induce differences in the charge gating on the sensitive SWCNT-FET. To our delight, unique signals were observed when the KF nanocircuit was incubated with 6-Cl-dGTP and poly(dC)₄₂. Positive ΔI(t) excursions, or “upswitches,” likely represent a different mode of enzyme closure (FIG. 2D). These signals are the opposite from the result of native dGTP incorporation.₂₀ As shown in the experiments with charge mutants of T4 lysozyme,₂₉ either negatively charged amino acid functional groups must move closer to the nanotube or positively charged functional groups must move further away during 6-Cl-dGTP incorporation. For the 2-thiosubstituted dNTPs, a complicated mixture of up- and downswitching events was observed. The random distribution of up- and down-switching associated with KF closures suggests the enzymes applies more than one conformation to maintain efficient catalysis.

Despite the large overlap in the kinetics for native and analog dGTP incorporation, KF clearly accesses different conformations during catalysis with 2-thio dCTP, 2-thio dTTP and 6-Cl-dGTP. These unique up-switching signals could be caused by conformational changes regulated by the KF J-helix during partitioning to and from the exonuclease domain, as KF recognizes incorporation of a less than perfectly complementary base.[14,30-32] However, such shuttling would need to occur at very fast rates, as no difference between enzyme closure times, τ_{closed}, were observed for 6-Cl-dGTP, 2-thio-dTTP, and 2-thio-dCTP compared to native dNTPs. The potential of these distinct signals to allow incoming dNTP discrimination deserve further study for possible applications in DNA sequencing.

Conclusion.

In summary, dNTP analogs challenge the limits of nucleotide incorporation by DNA polymerases, including the stereochemistry at the electrophilic phosphate, the hydrogen bonding capability of the incoming base, and the extent of enzyme closure. Since most analogs examined decrease τₒₚₑₙ and consequently the enzyme's rate, the rate-determining dNTP recognition step appears highly sensitive to minor variation in substrate structure. On the contrary, even dramatic substitutions at the reactive site of bond formation fail to impact KF closure times. The results described here suggest two possible strategies aimed at KF conformational assignment during each step of dNTP analog incorporation using the KF nanocircuit. First, modifications can target the conformation of the open KF during dNTP recognition and activation. To date, however, modified dNTPs including 6-Cl-dGTP and α-thio-dNTP can only slow the enzyme's average rate. Another strategy, which merits additional study, affects the closed conformation of KF. Modified bases, such as 6-Cl-dGTP, 2-thiodCTP and 2-thio-dTTP, can alter the conformation of the enzyme during incorporation, dramatically affecting the electronic signal observed as increases in current passing through the SWCNT. Such up-switching can be readily distinguished from incorporation of native dNTPs and provides direct observation of the enzyme readily accommodating unnatural dNTPs. In conclusion, conformationally sensitive electronic measurements of even a well-studied enzyme can reveal new and unexpected aspects of the enzyme's motions and dynamics.

### References (Example 1)

[1] Echols, H.; Goodman, M. F. Annu. Rev. Biochem. 1991, 60, 477; [2] Kunkel, T. A. J. Biol. Chem. 2004, 279, 16895; [3] Goodman, M. F. Proc. Natl. Acad. Sci. 1997, 94, 10493; [4] Kool, E. T. Annu. Rev. Biochem. 2002, 71, 191; [5] Betz, K.; Malyshev, D. A.; Lavergne, T.; Welte, W.; Diederichs, K.; Dwyer, T. J.; Ordoukhanian, P.; Romesberg, F. E.; Marx, A; Nat. Chem. Biol. 2012, 8, 612; [6] Burgers, P. M.; Eckstein, F. J. Biol. Chem. 1979, 254, 6889; [7] Chiaramonte, M.; Moore, C. L.; Kincaid, K.; Kuchta, R. D; Biochemistry 2003, 42, 10472; [8] Kim, T. W.; Delaney, J. C.; Essigmann, J. M.; Kool, E. T. Proc; Natl. Acad. Sci. U.S.A. 2005, 102, 15803; [9] Kincaid, K.; Beckman, J.; Zivkovic, A.; Halcomb, R. L.; Engels, J. W.; Kuchta, R. D. Nucleic Acids Res. 2005, 33, 2620; [10] Sintim, H. O.; Kool, E. T. J. Am. Chem. Soc. 2006, 128, 396; [11] Deniz, A. A.; Mukhopadhyay, S.; Lemke, E. A. J. R. Soc; Interface 2008, 5, 15; [12] Lu, H. P. Chem. Soc. Rev. 2014, 43, 1118; [13] Min, W.; English, B. P.; Luo, G.; Cherayil, B. J.; Kou, S. C.; Xie, X. S. Acc. Chem. Res. 2005, 38, 923; [14] Christian, T. D.; Romano, L. J.; Rueda, D. Proc. Natl. Acad. Sci; U.S.A. 2009, 106, 21109; [15] Santoso, Y.; Joyce, C. M.; Potapova, O.; Le Reste, L.; Hohlbein, J.; Torella, J. P.; Grindley, N. D. F.; Kapanidis, A. N. Proc; Natl. Acad. Sci. U.S.A. 2010, 107, 715; [16] Berezhna, S. Y.; Gill, J. P.; Lamichhane, R.; Millar, D. P. J. Am; Chem. Soc. 2012, 134, 11261; [17] Choi, Y.; Moody, I. S.; Sims, P. C.; Hunt, S. R.; Corso, B. L.; Perez, I.; Weiss, G. A.; Collins, P. G. Science 2012, 335, 319; [18] Choi, Y.; Moody, I. S.; Sims, P. C.; Hunt, S. R.; Corso, B. L.; Seitz, D. E.; Blaszczak, L. C.; Blaszcazk, L. C.; Collins, P. G.; Weiss, G. A. J. Am. Chem. Soc. 2012, 134, 2032; [19] Sims, P. C.; Moody, I. S.; Choi, Y.; Dong, C.; Iftikhar, M.; Corso, B. L.; Gul, O. T.; Collins, P. G.; Weiss, G. A. 2013; [20] Olsen, T. J.; Choi, Y.; Sims, P. C.; Gul, O. T.; Corso, B. L.; Dong, C.; Brown, W. A.; Collins, P. G.; Weiss, G. A. J. Am; Chem. Soc. 2013, 135, 7855; [21] Frey, M. W.; Sowers, L. C.; Millar, D. P.; Benkovic, S. J; Biochemistry 1995, 34, 9185; [22] Knowles, J. R. Annu. Rev. Biochem. 1980, 49, 877; [23] Bryant, F. R.; Johnson, K. A.; Benkovic, S. J. Biochemistry 1983, 22, 3537; [24] Eckstein, F.; Jovin, T. M. Biochemistry 1983, 22, 4546; [25] Mizrahi, V.; Henrie, R. N.; Marlier, J. F.; Johnson, K. A.; Benkovic, S. J. Biochemistry 1985, 24, 4010; [26] Patro, J. N.; Urban, M.; Kuchta, R. D. Biochemistry 2009, 48, 180; [27] Devadoss, B.; Lee, I.; Berdis, A. J. Biochemistry 2007, 46, 13752; [28] Zhang, X.; Motea, E.; Lee, I.; Berdis, A. J. Biochemistry 2010, 49, 3009; [29] Choi, Y.; Olsen, T. J.; Sims, P. C.; Moody, I. S.; Corso, B. L.; Dang, M. N.; Weiss, G. A.; Collins, P. G. Nano Lett. 2013, 13, 625; [30] Mizrahi, V.; Benkovic, P.; Benkovic, S. J. Proc. Natl. Acad. Sci; U.S.A. 1986, 83, 5769; [31] Joyce, C. J. Biol. Chem. 1989, 264, 10858; [32] Tuske, S.; Singh, K.; Kaushik, N.; Modak, M. J. J. Biol. Chem; 2000, 275, 23759;

### Example 2. Incorporation of Deoxynucleoside Triphosphate Analogs by Single-Molecule DNA Polymerase I (Klenow Fragment) Nanocircuits-2

Description.

Single copies of the Klenow Fragment (KF) of DNA polymerase I were attached to single-walled carbon nanotube devices and measured electrically in the presence of different chemical co-factors. All aspects of the fabrication followed the protocol described by Olsen et. al.

Results.

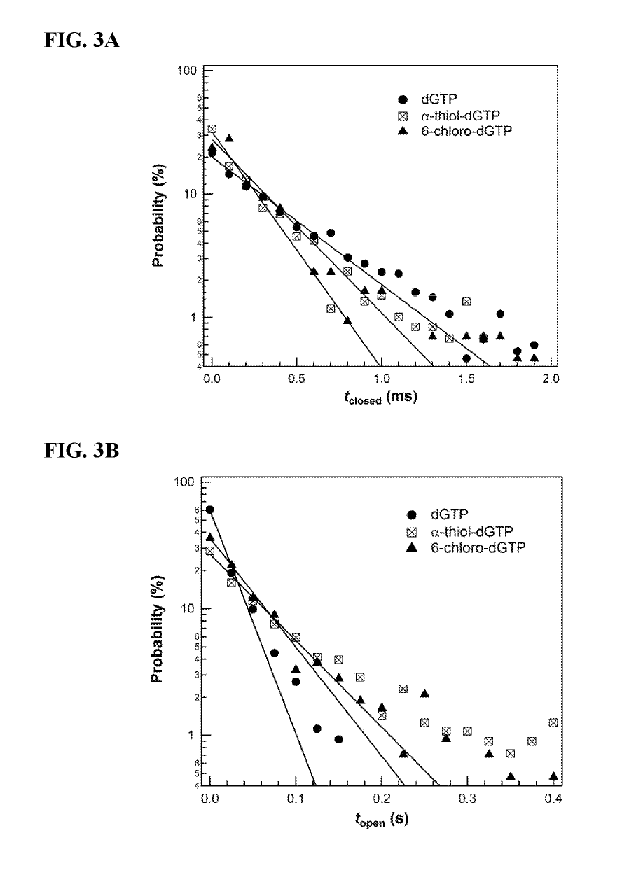

FIGS. 4A-4B demonstrate that when KF processes poly(dA)42 in the presence of the natural nucleotide deoxythymidine triphosphate (dTTP, each base pair incorporation produces a negative current spike ΔI<0. When dTTP is replaced by the unnatural nucleotide 2-thio-2′-deoxythimidine-5′-triphosphate (2-thio-dTTP), base incorporations produce positive current spikes ΔI>0.

FIG. 5A demonstrate that when KF processes heterogeneous substrates in the presence of all four natural nucleotides (dNTP), each base pair incorporation produces a negative current spike ΔI<O. Individual spikes can be enumerated as shown, but in general they do not differentiate one type of base from another.

As demonstrated in FIG. 5B, simulation of the same data set with dTTP replaced by 2-thio-dTTP. With the thiolated deoxythymidine, positive spikes now indicate (#2, 6, 7) the locations where T nucleotides were incorporated. This observation can be extended to RNA sequencing by replacing KF by an RNA polymerase. The process depicted in FIG. 5B identifies all of the T nucleotide incorporations in a particular DNA substrate. The process can be extended to all four bases by measuring the substrate with each of the four different thiolated nucleotides.

FIG. 5C demonstrate that when KF processes heterogeneous substrates in the presence of natural nucleotides (dNTP) mixed with certain analogs, the resulting pattern contains positive and negative current spikes that can be used to identify a chosen base. This example shows data acquired using three native nucleotides (dATP, dTTP, dCTP) mixed with 6-Cl-2APTP as an analog for G incorporations. Current spikes in this data set are numbered to enumerate 15 base incorporation events. Most of the events (#1, 4, 7, 9, 10, 13, 14, 15) consist of a single negative current spike that identifies incorporation of a native nucleotide. Five of the events (#2, 3, 6, 8, 11) are highlighted with arrows because they are positive current spikes that identify 6-Cl-2APTP nucleotide incorporations. Since 6-Cl-2APTP is used here as an analog of the native dGTP nucleotide, these five events identify G nucleotides in the DNA sequence.

Two of the events (#5, 12) shown in FIG. 5C contain a pair of closely-spaced current spikes. These pairs may indicate incorporation of one native and one 6-Cl-2APTP nucleotide in rapid succession. Alternately, a pair of spikes could be a unique signal resulting from 6-Cl-dAPTP acting as a pseudo-analog of dATP.

### References (Example 2 and Background)

[1] T. J. Olsen, Y. Choi, P. C. Sims, 0. T. GuI, B. L. Corso, C. Dong, . . . G. A. Weiss, Electronic Measurements of Single-Molecule Processing by DNA polymerase I (Klenow fragment), I Am. Chem. Soc. 135, 7855 (2013); [2] Y. Choi, 1. S. Moody, P. C. Sims, S. R. Hunt, B. L. Corso, G. A. Weiss, and P. G. Collins, Single-Molecule Lysozyme Dynamics Monitored by an Electronic Circuit, Science 335, 319 (2012); [3]. Y. Choi, 1. 5. Moody, P. C. Sims, S. R. Hunt, B. L. Corso, D. E. Seitz, . . . G. A. Weiss, Single Molecule Dynamics of Lysozyme Processing Distinguishes Linear and Cross-linked Peptidoglycan Substrates, 1. Am. Chem. Soc. 134, 2032 (2012); [4]. Y. Choi, T. J. Olsen, P. C. Sims, 1. S. Moody, B. L. Corso, M. N. Dang, P. G. Collins, Dissecting Single-Molecule Signal Transduction in Carbon Nanotube Circuits with Protein Engineering, Nano Lett. 13, 625 (2013); [5]. P. C. Sims, I. S. Moody, Y. Choi, C. Dong, M. Iftikhar, B. L. Corso, . . . G. A. Weiss, Electronic Measurements of Single-Molecule Processing by protein kinase A, I Ani. Chem. Soc. 135, 7861 (2013; [6]. L. T. C. Franca, E. Carrilho, and T. B. L. Kist, A review of DNA sequencing techniques, Quarterly Reviews of Biophysics 35, 169 (2002); [7]. 5. E. Jacutin, Unnatural Nucleotides for DNA Sequencing. (Texas A & M University, College Station, Tex., 1997). [8]. T. D. Harris, P. R. Buzby, H. Babcock, E. Beer, J. Bowers, I. Braslaysky, Z. Xie, Single-Molecule DNA Sequencing of a Viral Genome Science 320 106 (2008); [9]. D. Stoddart, A. J. Heron, E. Mikhailova, G. Maglia, and H. Bayley, Single-nucleotide discrimination in immobilized DNA oligonucleotides with a biological nanopore, Proc. Natl. Acad. Sci. U.S.A. 106, 7702 (2009); [10]. S. Sorgenfrei, C.-y. Chiu, R. L. Gonzalez, Y.-J. Yu, P. Kim, C. Nuckolls, and K. L. Shepard, Label-free single-molecule detection of DNA-hybridization kinetics with a carbon nanotube field-effect transistor, Nat. Nanotechnol. 6, 126 (2011); [11]. T. C. Glenn, Field guide to next-generation DNA sequencers, Molecular Ecology Resources 11, 759 (2011).

### Example 3: Expression and Purification of KF

Reagents purchased commercially include antibiotics (Fisher Scientific), Ni-IMAC resin (Bio-Rad Laboratories), cell lines (Stratagene), deoxynucleoside triphosphates (Fisher Scientific), deoxynucleoside triphosphate analogs (Trilink Biotechnologies), enzymes (New England Biolabs or Fermentas), oligonucleotides (Fisher), high-resolution agarose (The Nest Group) and 96-well fluorescence plates (Nunc). All other chemicals were purchased commercially from Acros Organics, EMD, Fisher Scientific, or Sigma Aldrich. All reagents were used as received.

A pET28c plasmid containing a gene encoding KF(D355A/E357A/C907S/L790C),^{1,2} referred to hereafter as KF, was used to transform CaCl₂-competent BL21(DE3) E. coli cells by heat shock. Following overnight growth on solid media, a single colony was used to inoculate 25 mL LB media supplemented with 40 ng/mL kanamycin for growth in liquid media overnight at 37° C. with shaking. LB (1 L) supplemented with 40 ng/mL kanamycin was inoculated with 10 mL of the overnight culture and incubated with shaking at 37° C. for several hours. Once the cells reached late log phage (OD₆₀₀=0.9), KF expression was induced by the addition of 1 mM IPTG. After 3-4 h of protein expression at 37° C. with shaking, cells were harvested by centrifugation (6000 rpm, 20 min, 4° C.) and resuspended in lysis buffer (20 mM Tris, 50 mM NaCl, 10 mM BME, pH 8.0). Cells were lysed by sonication and the cell debris was collected by centrifugation (15,000 rpm, 45 min, 4° C.). Following filtration through a 0.45 μm pore filter, the lysate supernatant was allowed to bind to Ni-IMAC resin overnight at 4° C. KF was eluted in the lysis buffer with 250 mM imidazole, concentrated, and then treated with TEV protease for two days at 4° C. The mixture was centrifuged and then filtered through a 0.45 μm filter prior to size exclusion chromatography in TBS (20 mM Tris, 50 mM NaCl, 100 μM TCEP, pH 7.9) on a Bio-Rad Biologic DuoFlow FPLC. KF purity was assessed by SDS-PAGE (FIG. 6).

Ensemble Activity of KF and dNTP Analog Incorporation

Oligonucleotides Used to Test Activity

Table 2 lists the oligonucleotides used to test KF activity, dNTP analog incorporation, and for measurements with the nanocircuit. Upon receipt, HPLC-purified oligonucleotides were solubilized in water to 100 μM. Bold regions indicate the M13 priming site. Italicized regions indicate restriction sites. [2AmPur] indicates 2-aminopurine.

TABLE 2
Oligonucleotides used for activity and electronic measurements

| Oligonucleotide | Sequence | Use |
|---|---|---|
| M13F | TGTAAAACGACGGCCAGT | M13F sequence primer |
| | [SEQ ID NO: 1] | |
| ActAssay | TCGAGCTATCTCTAAAGC[2AmPur] | Ensemble activity assay |
| Template | GCTAACTATCGAGCTATCGCGAAA | template containing 2- |
| | CTGGCCGTCGTTTTACA | aminopurine |
| | [SEQ ID NO: 2] | |
| A/T Incorporation | CCTAACGCAGATAGACGTTGTTTA | Test incorporation of dATP or |
| Assay Template | GAGCCCGGGTCGGCCATACTGGC | dTTP analogs in FIG. S3a |
| | CGTCGTTTTACA | |
| | [SEQ ID NO: 3] | |
| G/C Incorporation | CCTAACGCAGATAGACGTTGTTTA | Test incorporation of dCTP or |
| Assay Template | GAGATTTAAATTCGGCCACTGGC | dGTP analogs in FIG. S3b |
| | CGTCGTTTTACA | |
| | [SEQ ID NO: 4] | |
| poly(dA)₄₂ | AAAAAAAAAAAAAAAAAAAA | Test native and analog dTTP |
| | AAAAAAAAAAAAAAAAAAAA | incorporation on nanocircuit |
| | AAACTGGCCGTCGTTTTACA | |
| | [SEQ ID NO: 5] | |
| poly(dT)₄₂ | TTTTTTTTTT TTTTTTTTTT | Test native and analog dATP |
| | TTTTTTTTTT TTTTTTTTTT | incorporation on nanocircuit |
| | TTACTGGCCGTCGTTTTACA | |
| | [SEQ ID NO: 6] | |
| poly(dG)₄₂ | GGGGGGGGGG GGGGGGGGGG | Test native and analog dCTP |
| | GGGGGGGGGG GGGGGGGGGG | incorporation on nanocircuit |
| | GGACTGGCCGTCGTTTTACA | |
| | [SEQ ID NO: 7] | |
| poly(dC)₄₂ | CCCCCCCCCC CCCCCCCCCC | Test native and analog dGTP |
| | CCCCCCCCCC CCCCCCCCCC | incorporation on nanocircuit |
| | CCACTGGCCGTCGTTTTACA | |
| | [SEQ ID NO: 8] | |

### Ensemble Assay for KF Activity

To confirm activity of the variant KF(L790C) versus wild-type KF, a previously described assay was adapted as follows.^{1,3} A randomized DNA template containing both 2-aminopurine (ActAssay template in Table S1) and an M13 priming site (underlined) was annealed to the M13F primer by heating the mixture to 65° C. and slow-cooling to room temperature for 1 h. A comparable decrease in fluorescence was observed for KF(L790C) and wild-type KF (both 1 μM) upon incubation with the primer-template mixture (25 μM) and dNTPs (250 μM). The raw fluorescence data was corrected by subtraction of background, which was measured in the absence of dNTPs. The excitation and emission wavelengths employed in this experiment were 305 and 365 nm, respectively.

Ensemble Assay for dNTP Analog Incorporation

To confirm incorporation of dNTP analogs, randomized DNA templates (Table S1) were polymerized by KF after hybridization to an M13F primer. Positive control reactions contained KF (1 μM), dNTPs or dNTP analogs (100 μM), and A/T or G/C incorporation template-primer (5 μM) in 10 mM Tris, 50 mM NaCl, 10 mM MgCl₂, 1 mM DTT, pH 7.9. Reactions to test dNTP analog incorporation contained 100 μM analog in place of its native dNTP, and negative control reactions omitted either the analog or its native dNTP. Reactions were kept at 25° C. for 2 h in a thermal cycler before electrophoresis on a 5% high resolution agarose gel (FIG. 8B).

### Embodiments of the Present Disclosure

A method of detecting a change in a nucleic acid polymerase conformation, the method comprising:
(i) contacting a nucleic acid polymerase non-covalently attached to a single walled carbon nanotube (SWNT) with a first nucleotide or first nucleotide analog and a template nucleic acid sequence thereby forming a conformationally changed nucleic acid polymerase bound to the first nucleotide or the first nucleotide analog and the template nucleic acid sequence;
(ii) detecting the conformationally changed nucleic acid polymerase by measuring a first electrical conductance change in the SWNT between the nucleic acid polymerase and the conformationally changed nucleic acid polymerase.

The method wherein said nucleic acid polymerase is contacted with a first nucleotide analog.

The method further comprising (iii) identifying said first nucleotide or first nucleotide analog based on a first signal produced by said first electrical conductance change.

The method further comprising: (iv) allowing said conformationally changed nucleic acid polymerase to release said first nucleotide or first nucleotide analog thereby reforming said nucleic acid polymerase.

The method further comprising
(v) contacting a nucleic acid polymerase non-covalently attached to a single walled carbon nanotube (SWNT) with a second nucleotide or second nucleotide analog and said template nucleic acid sequence thereby forming a conformationally changed nucleic acid polymerase bound to the second nucleotide or the second nucleotide analog and the template nucleic acid sequence; and
(vi) detecting the conformationally changed nucleic acid polymerase by measuring an electrical conductance change in the SWNT between the nucleic acid polymerase and the conformationally changed nucleic acid polymerase bound to the second nucleotide or the second nucleotide analog and the template nucleic acid sequence.

The method of claim 5, wherein said nucleic acid polymeraseis contacted with a second nucleotide analog.

The method further comprising (vii) identifying said second nucleotide or second nucleotide analog based on a second signal produced by said second electrical conductance change; and identifying a sequence within the template nucleic acid.

The method wherein said first nucleotide analog or said second nucleotide analog hybridizes to said template nucleic acid sequence with non-Watson-Crick base pairing.

The method wherein said first nucleotide analog or said second nucleotide analog is 2-thio dCTP, 2-thio dTTP, or 6-Cl-dGTP, 6-aza-dTTP, α-thio-dATP, or α-thio-dTTP.

The method wherein said first nucleotide analog or said second nucleotide analog is modified at the triphosphate moiety.

The method wherein said triphosphate moiety comprises an α-thio substitution.

The method wherein said first nucleotide analog or said second nucleotide analog is α-thio-dATP, α-thio-dGTP, α-thio-dCTP, or α-thio-dTTP.

The method wherein said first nucleotide analog or said second nucleotide analog further comprises a substitution at the nucleobase.

The method wherein said first nucleotide analog or said second nucleotide analog is α-thio-2-thio-dTTP, α-thio-2-thio-dCTP, α-thio-6-Cl-20APTP, or 6-Cl-2APTP.

1. A method of detecting a change in a nucleic acid polymerase conformation, the method comprising:
(i) contacting a nucleic acid polymerase non-covalently attached to a single walled carbon nanotube (SWNT) with a first nucleotide or first nucleotide analog and a template nucleic acid sequence thereby forming a conformationally changed nucleic acid polymerase bound to the first nucleotide or the first nucleotide analog and the template nucleic acid sequence;
(ii) detecting the conformationally changed nucleic acid polymerase by measuring a first electrical conductance change in the SWNT between the nucleic acid polymerase and the conformationally changed nucleic acid polymerase.

2. The method of claim 1, wherein said nucleic acid polymerase is contacted with a first nucleotide analog.

3. The method of claim 1, further comprising (iii) identifying said first nucleotide or first nucleotide analog based on a first signal produced by said first electrical conductance change.

4. The method of claim 3, further comprising: (iv) allowing said conformationally changed nucleic acid polymerase to release said first nucleotide or first nucleotide analog thereby reforming said nucleic acid polymerase.

5. The method of claim 4, further comprising
(v) contacting a nucleic acid polymerase non-covalently attached to a single walled carbon nanotube (SWNT) with a second nucleotide or second nucleotide analog and said template nucleic acid sequence thereby forming a conformationally changed nucleic acid polymerase bound to the second nucleotide or the second nucleotide analog and the template nucleic acid sequence; and
(vi) detecting the conformationally changed nucleic acid polymerase by measuring an electrical conductance change in the SWNT between the nucleic acid polymerase and the conformationally changed nucleic acid polymerase bound to the second nucleotide or the second nucleotide analog and the template nucleic acid sequence.

6. The method of claim 5, wherein said nucleic acid polymeraseis contacted with a second nucleotide analog.

7. The method of claim 5, further comprising (vii) identifying said second nucleotide or second nucleotide analog based on a second signal produced by said second electrical conductance change; and identifying a sequence within the template nucleic acid.

8. The method of claim 7, wherein said first nucleotide analog or said second nucleotide analog hybridizes to said template nucleic acid sequence with non-Watson-Crick base pairing.

9. The method of claim 7, wherein said first nucleotide analog or said second nucleotide analog is 2-thio dCTP, 2-thio dTTP, or 6-Cl-dGTP, 6-aza-dTTP, α-thio-dATP, or α-thio-dTTP.

10. The method of claim 7, wherein said first nucleotide analog or said second nucleotide analog is modified at the triphosphate moiety.

11. The method of claim 10, wherein said triphosphate moiety comprises an α-thio substitution.

12. The method of claim 11, wherein said first nucleotide analog or said second nucleotide analog is α-thio-dATP, α-thio-dGTP, α-thio-dCTP, or α-thio-dTTP.

13. The method of claim 10, wherein said first nucleotide analog or said second nucleotide analog further comprises a substitution at the nucleobase.

14. The method of claim 13, wherein said first nucleotide analog or said second nucleotide analog is α-thio-2-thio-dTTP, α-thio-2-thio-dCTP, α-thio-6-Cl-20APTP, or 6-Cl-2APTP.


SEQUENCE LISTING (12 sequences)

SEQ ID NO: 1 - DNA, 18 bp, Artificial Sequence
  Synthetic polynucleotide
tgtaaaacgacggccagt

SEQ ID NO: 2 - DNA, 60 bp, Artificial Sequence
  Synthetic polynucleotide
  modified_base (19)..(19): 2-Aminopurine
tcgagctatctctaaagcagctaactatcgagctatcgcgaaactggccgtcgttttaca

SEQ ID NO: 3 - DNA, 59 bp, Artificial Sequence
  Synthetic polynucleotide
cctaacgcagatagacgttgtttagagcccgggtcggccatactggccgtcgttttaca

SEQ ID NO: 4 - DNA, 59 bp, Artificial Sequence
  Synthetic polynucleotide
cctaacgcagatagacgttgtttagagatttaaattcggccactggccgtcgttttaca

SEQ ID NO: 5 - DNA, 60 bp, Artificial Sequence
  Synthetic polynucleotide
aaaaaaaaaaaaaaaaaaaaaaaaaaaaaaaaaaaaaaaaaaactggccgtcgttttaca

SEQ ID NO: 6 - DNA, 60 bp, Artificial Sequence
  Synthetic polynucleotide
ttttttttttttttttttttttttttttttttttttttttttactggccgtcgttttaca

SEQ ID NO: 7 - DNA, 60 bp, Artificial Sequence
  Synthetic polynucleotide
ggggggggggggggggggggggggggggggggggggggggggactggccgtcgttttaca

SEQ ID NO: 8 - DNA, 60 bp, Artificial Sequence
  Synthetic polynucleotide
ccccccccccccccccccccccccccccccccccccccccccactggccgtcgttttaca

SEQ ID NO: 9 - DNA, 42 bp, Artificial Sequence
  Synthetic polynucleotide
tttttttttttttttttttttttttttttttttttttttttt

SEQ ID NO: 10 - DNA, 42 bp, Artificial Sequence
  Synthetic polynucleotide
aaaaaaaaaaaaaaaaaaaaaaaaaaaaaaaaaaaaaaaaaa

SEQ ID NO: 11 - DNA, 42 bp, Artificial Sequence
  Synthetic polynucleotide
gggggggggggggggggggggggggggggggggggggggggg

SEQ ID NO: 12 - DNA, 42 bp, Artificial Sequence
  Synthetic polynucleotide
cccccccccccccccccccccccccccccccccccccccccc